(19) United States
(12) Patent Application Publication
(10) Pub. No.: US 2025/0268801 A1
(43) Pub. Date: Aug. 28, 2025

(54) DRY CREAMS AND TOPICAL DELIVERY SYSTEMS

(71) Applicant: UEA Enterprises Limited, Norfolk (GB)

(72) Inventors: Sheng QI, Norfolk (GB); Pratchaya TIPDUANGTA, Norfolk (GB); Yan Fen LEE, Norfolk (GB); Xin Yi TEOH, Norfolk (GB)

(73) Assignee: UEA Enterprises Limited, Norfolk, EN (GB)

(21) Appl. No.: 18/696,137

(22) PCT Filed: Sep. 29, 2022
(86) PCT No.: PCT/EP2022/077221

§ 371 Date: Mar. 27, 2024

(30) Foreign Application Priority Data
Sep. 29, 2021 (GB) 2113926.6

(51) Int. Cl. A61K 8/02; A61Q 19/00

(52) U.S. Cl. CPC A61K 8/0208; A61Q 19/00; A61K 2800/10

(57) ABSTRACT

The present invention relates to highly absorbable nanofibrous and microfibrous polymeric materials effective in delivering bioactives or pharmaceutical active ingredients topically, for both cosmetic and pharmaceutical applications.

### FIELD OF THE INVENTION

The present invention relates to highly absorbable nanofibrous and microfibrous polymeric materials effective in delivering bioactives or pharmaceutical active ingredients topically, for both cosmetic and pharmaceutical applications.

### BACKGROUND OF THE INVENTION

There is increasing need for effective topical delivery of bioactives, pharmaceuticals and other agents. One particular area of focus is the cosmetic and personal care fields. A further area of focus is healthcare.

Cosmetics and personal care products have played an important part in our lives for centuries. In recent times, there has been a surge in cosmetic sale and this trend is set to continue rising.

Cosmetic and personal care products rely heavily on customer acceptability of the products. A product's physical appearance, feel and ease of use are essential for its ultimate success. Furthermore, the incorporation of vitamins, nutrients, antioxidants and other bioactives into cosmetic products are becoming important differentiation strategies.

Beauty masks are one of the branches of beauty products that have shown strong increases in sales globally in recent years. Conventional beauty face masks available in the market are mainly cellulose masks that are pre-moistened with skin nutrients. In 2013, beauty masks alone recorded sales of $800m in China, with sales expected to reach $130 billion by 2019. Furthermore, with an increasing number of products aimed at special target audiences, there is increasing significance in groups that have previously not received as much attention.

WO2009031620 and WO2014098764 both describe face masks made from electrospun polymeric materials. The face masks are designed to dissolve on contact with water (for example moisture contained on a user's face).

Topical delivery is also important within healthcare. Topical formulations for pharmaceutical delivery are becoming increasingly popular. Topical delivery has a number of advantages: the ability to deliver drug substance more selectively to a specific site, avoiding fluctuations in drug levels, inter- and intra-patient variations, improved compliance, and an enhanced suitability for self-medication. Skin provides an ideal site for the delivery of drug substances for both local and systemic effects. With topical therapies, the formulation and delivery vehicle as a whole is as important as the bioactive agent itself, because the interaction of the delivery vehicle can alter the effects of the penetrating agent. Today, in addition to the range of drugs applied for local effects in the skin and associated tissues, nearly twenty drugs have been successfully developed for transdermal delivery using various topical dosage forms such as patches, gels and ointments and cutaneous solutions.

There remains a growing need for more effective dry creams and topical delivery systems.

### SUMMARY OF THE INVENTION

In a first aspect of the present invention there is provided a dry cream for delivery of oil(s) to a user, said dry cream comprising a mat of electrospun hydrophilic polymeric fibers containing said oil(s).

There are many advantages associated with a dry cream over traditional creams, including: a reduction in weight and bulk, since water is often the highest weightage in the formulation; environmental sustainability in terms of lower transportation costs in view of a lighter and simpler product, less packaging; emulsification under mixing with water, whereby the cream may become fully absorbed after rubbing into skin; reduction/complete elimination of preservatives, since the removal of water/moisture reduces/eliminates the need for preservatives to combat the growth of microorganisms; and superior storage qualities given that no phase separation can occur in the dried state; prolonged shelf-life due to the minimal content of water. Without wishing to be bound by theory, it is believed that the presence of the polymeric fibers prepared using electrospinning in conjunction with the oil(s) in the dry cream suppresses the dry cream from undergoing phase separation and/or sedimentation.

Preferably, in the dry cream as described herein, the dry cream comprises less than 2.0 wt % of water, less than 1.9 wt % of water, less than 1.8 wt % of water, less than 1.7 wt % of water, less than 1.6 wt % of water, less than 1.5 wt % of water, less than 1.4 wt % of water, less than 1.3 wt % of water, less than 1.2 wt % of water, less than 1.1 wt % of water, less than 1.0 wt % of water, less than 0.9 wt % of water, less than 0.8 wt % of water, less than 0.7 wt % of water, less than 0.6 wt % of water, less than 0.5 wt % of water, less than 0.4 wt % of water, less than 0.3 wt % of water, less than 0.2 wt % of water, less than 0.1 wt % of water, less than 0.09 wt % of water, less than 0.08 wt % of water, less than 0.07 wt % of water, less than 0.06 wt % of water, less than 0.05 wt % of water, less than 0.04 wt % of water, less than 0.03 wt % of water, less than 0.02 wt % of water, or less than 0.01 wt % of water, based on a total weight of the dry cream.

Preferably, in the dry cream as described herein, the total polymeric fiber content is about 40 wt % to about 99 wt %, about 42 wt % to about 98 wt %, about 44 wt % to about 97 wt %, about 46 wt % to about 96.5 wt %, about 48 wt % to about 96.5 wt %, or about 50 wt % to about 96.5 wt %, based on a total weight of the dry cream.

Preferably, in the dry cream as described herein, the total oil content of the dry cream is about 1 wt % to about 35 wt %, about 1 wt % to about 30 wt %, about 1 wt % to about 25 wt %, about 1 wt % to about 20 wt %, about 1 wt % to about 15 wt %, about 1 wt % to about 10 wt %, about 2 wt % to about 8 wt %, about 3 wt % to about 6 wt %, or about 3.5 wt % to about 5 wt %, based on a total weight of the dry cream.

Preferably, in the dry cream as described herein, the total oil content of the dry cream is about 1 wt % to about 30 wt %, about 1 wt % to about 25 wt %, about 1 wt % to about 20 wt %, about 1 wt % to about 15 wt %, about 1 wt % to about 10 wt %, about 2 wt % to about 8 wt %, about 3 wt % to about 6 wt %, or about 3.5 wt % to about 5 wt %, based on a total weight of the dry cream; and the dry cream comprises less than 2.0 wt % of water, less than 1.9 wt % of water, less than 1.8 wt % of water, less than 1.7 wt % of water, less than 1.6 wt % of water, less than 1.5 wt % of water, less than 1.4 wt % of water, less than 1.3 wt % of water, less than 1.2 wt % of water, less than 1.1 wt % of water, less than 1.0 wt % of water, less than 0.9 wt % of water, less than 0.8 wt % of water, less than 0.7 wt % of water, less than 0.6 wt % of water, less than 0.5 wt % of water, less than 0.4 wt % of water, less than 0.3 wt % of water, less than 0.2 wt % of water, less than 0.1 wt % of water, less than 0.09 wt % of water, less than 0.08 wt % of water, less than 0.07 wt % of water, less than 0.06 wt % of water, less than 0.05 wt % of water, less than 0.04 wt % of water, less than 0.03 wt % of water, less than 0.02 wt % of water, or less than 0.01 wt % of water, based on a total weight of the dry cream.

Preferably, in the dry cream as described herein, the total oil content of the dry cream is about 1 wt % to about 25 wt %, about 1 wt % to about 20 wt %, about 1 wt % to about 15 wt %, about 1 wt % to about 10 wt %, about 2 wt % to about 8 wt %, about 3 wt % to about 6 wt %, or about 3.5 wt % to about 5 wt %, based on a total weight of the dry cream; and the dry cream comprises less than 2.0 wt % of water, less than 1.9 wt % of water, less than 1.8 wt % of water, less than 1.7 wt % of water, less than 1.6 wt % of water, less than 1.5 wt % of water, less than 1.4 wt % of water, less than 1.3 wt % of water, less than 1.2 wt % of water, less than 1.1 wt % of water, less than 1.0 wt % of water, less than 0.9 wt % of water, less than 0.8 wt % of water, less than 0.7 wt % of water, less than 0.6 wt % of water, less than 0.5 wt % of water, less than 0.4 wt % of water, less than 0.3 wt % of water, less than 0.2 wt % of water, less than 0.1 wt % of water, less than 0.09 wt % of water, less than 0.08 wt % of water, less than 0.07 wt % of water, less than 0.06 wt % of water, less than 0.05 wt % of water, less than 0.04 wt % of water, less than 0.03 wt % of water, less than 0.02 wt % of water, or less than 0.01 wt % of water, based on a total weight of the dry cream.

Preferably, in the dry cream as described herein, the total oil content of the dry cream is about 1 wt % to about 20 wt %, about 1 wt % to about 15 wt %, about 1 wt % to about 10 wt %, about 2 wt % to about 8 wt %, about 3 wt % to about 6 wt %, or about 3.5 wt % to about 5 wt %, based on a total weight of the dry cream; and the dry cream comprises less than 2.0 wt % of water, less than 1.9 wt % of water, less than 1.8 wt % of water, less than 1.7 wt % of water, less than 1.6 wt % of water, less than 1.5 wt % of water, less than 1.4 wt % of water, less than 1.3 wt % of water, less than 1.2 wt % of water, less than 1.1 wt % of water, less than 1.0 wt % of water, less than 0.9 wt % of water, less than 0.8 wt % of water, less than 0.7 wt % of water, less than 0.6 wt % of water, less than 0.5 wt % of water, less than 0.4 wt % of water, less than 0.3 wt % of water, less than 0.2 wt % of water, less than 0.1 wt % of water, less than 0.09 wt % of water, less than 0.08 wt % of water, less than 0.07 wt % of water, less than 0.06 wt % of water, less than 0.05 wt % of water, less than 0.04 wt % of water, less than 0.03 wt % of water, less than 0.02 wt % of water, or less than 0.01 wt % of water, based on a total weight of the dry cream.

Preferably, in the dry cream as described herein, the total oil content of the dry cream is about 1 wt % to about 15 wt %, about 1 wt % to about 10 wt %, about 2 wt % to about 8 wt %, about 3 wt % to about 6 wt %, or about 3.5 wt % to about 5 wt %, based on a total weight of the dry cream; and the dry cream comprises less than 2.0 wt % of water, less than 1.9 wt % of water, less than 1.8 wt % of water, less than 1.7 wt % of water, less than 1.6 wt % of water, less than 1.5 wt % of water, less than 1.4 wt % of water, less than 1.3 wt % of water, less than 1.2 wt % of water, less than 1.1 wt % of water, less than 1.0 wt % of water, less than 0.9 wt % of water, less than 0.8 wt % of water, less than 0.7 wt % of water, less than 0.6 wt % of water, less than 0.5 wt % of water, less than 0.4 wt % of water, less than 0.3 wt % of water, less than 0.2 wt % of water, less than 0.1 wt % of water, less than 0.09 wt % of water, less than 0.08 wt % of water, less than 0.07 wt % of water, less than 0.06 wt % of water, less than 0.05 wt % of water, less than 0.04 wt % of water, less than 0.03 wt % of water, less than 0.02 wt % of water, or less than 0.01 wt % of water, based on a total weight of the dry cream.

Preferably, in the dry cream as described herein, the total oil content of the dry cream is about 1 wt % to about 10 wt %, about 2 wt % to about 8 wt %, about 3 wt % to about 6 wt %, or about 3.5 wt % to about 5 wt %, based on a total weight of the dry cream; and the dry cream comprises less than 2.0 wt % of water, less than 1.9 wt % of water, less than 1.8 wt % of water, less than 1.7 wt % of water, less than 1.6 wt % of water, less than 1.5 wt % of water, less than 1.4 wt % of water, less than 1.3 wt % of water, less than 1.2 wt % of water, less than 1.1 wt % of water, less than 1.0 wt % of water, less than 0.9 wt % of water, less than 0.8 wt % of water, less than 0.7 wt % of water, less than 0.6 wt % of water, less than 0.5 wt % of water, less than 0.4 wt % of water, less than 0.3 wt % of water, less than 0.2 wt % of water, less than 0.1 wt % of water, less than 0.09 wt % of water, less than 0.08 wt % of water, less than 0.07 wt % of water, less than 0.06 wt % of water, less than 0.05 wt % of water, less than 0.04 wt % of water, less than 0.03 wt % of water, less than 0.02 wt % of water, or less than 0.01 wt % of water, based on a total weight of the dry cream.

Preferably, in the dry cream as described herein, the total oil content of the dry cream is about 2 wt % to about 8 wt %, about 3 wt % to about 6 wt %, or about 3.5 wt % to about 5 wt %, based on a total weight of the dry cream; and the dry cream comprises less than 2.0 wt % of water, less than 1.9 wt % of water, less than 1.8 wt % of water, less than 1.7 wt % of water, less than 1.6 wt % of water, less than 1.5 wt % of water, less than 1.4 wt % of water, less than 1.3 wt % of water, less than 1.2 wt % of water, less than 1.1 wt % of water, less than 1.0 wt % of water, less than 0.9 wt % of water, less than 0.8 wt % of water, less than 0.7 wt % of water, less than 0.6 wt % of water, less than 0.5 wt % of water, less than 0.4 wt % of water, less than 0.3 wt % of water, less than 0.2 wt % of water, less than 0.1 wt % of water, less than 0.09 wt % of water, less than 0.08 wt % of water, less than 0.07 wt % of water, less than 0.06 wt % of water, less than 0.05 wt % of water, less than 0.04 wt % of water, less than 0.03 wt % of water, less than 0.02 wt % of water, or less than 0.01 wt % of water, based on a total weight of the dry cream.

Preferably, in the dry cream as described herein, the total oil content of the dry cream is about 3 wt % to about 6 wt %, or about 3.5 wt % to about 5 wt %, based on a total weight of the dry cream; and the dry cream comprises less than 2.0 wt % of water, less than 1.9 wt % of water, less than 1.8 wt % of water, less than 1.7 wt % of water, less than 1.6 wt % of water, less than 1.5 wt % of water, less than 1.4 wt % of water, less than 1.3 wt % of water, less than 1.2 wt % of water, less than 1.1 wt % of water, less than 1.0 wt % of water, less than 0.9 wt % of water, less than 0.8 wt % of water, less than 0.7 wt % of water, less than 0.6 wt % of water, less than 0.5 wt % of water, less than 0.4 wt % of water, less than 0.3 wt % of water, less than 0.2 wt % of water, less than 0.1 wt % of water, less than 0.09 wt % of water, less than 0.08 wt % of water, less than 0.07 wt % of water, less than 0.06 wt % of water, less than 0.05 wt % of water, less than 0.04 wt % of water, less than 0.03 wt % of water, less than 0.02 wt % of water, or less than 0.01 wt % of water, based on a total weight of the dry cream.

Preferably, in the dry cream as described herein, the total oil content of the dry cream is about 3.5 wt % to about 5 wt %, based on a total weight of the dry cream; and the dry cream comprises less than 2.0 wt % of water, less than 1.9 wt % of water, less than 1.8 wt % of water, less than 1.7 wt % of water, less than 1.6 wt % of water, less than 1.5 wt % of water, less than 1.4 wt % of water, less than 1.3 wt % of water, less than 1.2 wt % of water, less than 1.1 wt % of water, less than 1.0 wt % of water, less than 0.9 wt % of water, less than 0.8 wt % of water, less than 0.7 wt % of water, less than 0.6 wt % of water, less than 0.5 wt % of water, less than 0.4 wt % of water, less than 0.3 wt % of water, less than 0.2 wt % of water, less than 0.1 wt % of water, less than 0.09 wt % of water, less than 0.08 wt % of water, less than 0.07 wt % of water, less than 0.06 wt % of water, less than 0.05 wt % of water, less than 0.04 wt % of water, less than 0.03 wt % of water, less than 0.02 wt % of water, or less than 0.01 wt % of water, based on a total weight of the dry cream.

Preferably, in the dry cream as described herein, a w/w ratio of the total oil content to the total polymeric fiber content is from about 1:5 to about 1:50, about 1:6 to about 1:45, about 1:7 to about 1:40, about 1:8 to about 1:35, about 1:9 to about 1:30, or about 1:10 to about 1:25.

Preferably, in the dry cream as described herein, the dry cream comprises one or more surfactant(s).

Preferably, in the dry cream as described herein, a w/w ratio of the total polymeric fiber content to the total surfactant content may be from about 1:5 to about 1:0.5, about 1:4 to about 1:0.6, about 1:3 to about 1:0.7, about 1:2 to about 1:0.8, or about 1:1 to about 1:0.9.

Preferably, in the dry cream as described herein, a w/w ratio of the total oil content to the total surfactant content may be from about 1:2 to about 1:20, about 1:3 to about 1:18, about 1:4 to about 1:16, about 1:5 to about 1:14, about 1:6 to about 1:12, about 1:7 to about 1:10, or about 1:8 to about 1:9.

Preferably, in the dry cream as described herein, the dry cream may comprise bioactive agent(s). The dry cream may be for delivery of oil(s) and bioactive agent(s) to a user.

In a further aspect of the present invention there is provided a topical delivery system for delivery of bioactive agent(s) to a user, said topical delivery system comprising a mat of electrospun hydrophilic polymeric fibers containing said bioactive agent(s), wherein said mat is configured to substantially retain its shape on a mounting surface when exposed to a wetting agent but disintegrate under mechanical agitation to form a cream or gel.

The topical delivery systems of the present invention have advantageous properties over prior art systems. They are able to retain their shape and form on a mounting surface when exposed to a wetting agent. Thus, they do not dissolve on contact with the wetting agent, for example water. This is advantageous over mats that do dissolve in contact with wetting agents, as this may allow for a more controlled delivery of bioactive agent(s) in a desired area. The higher structural integrity of these mats when exposed to wetting agents on the mounting surface compared to mats that dissolve directly into the wetting agent allows better adherence to the skin, meaning that the wetted mat is less likely to “drip” off the user; thus, a backing layer is not necessarily required and the topical delivery system can be held, for example, on the hand of the user after wetting but prior to application. Upon mechanical agitation the mat can uniformly disintegrate to form a cream or gel. Thus, the topical delivery systems according to an aspect of the present invention can be applied in one state (in a wetted state on the mounted surface containing bioactive agent(s)) and converted into a further state (a cream or gel) upon mechanical agitation by the user.

Topical delivery systems according to the present invention may be stored in a dried state to enhance shelf life and/or improve bioactive properties and then wetted for use. The topical delivery system may hold polar excipients, non-polar excipients or emulsions, including suspensions. The topical delivery system retains its shape during use but will disintegrate under mechanical agitation. The topical delivery system does not necessarily require polymer backing materials or the like to allow for complete disintegration under mechanical agitation. However, in some aspects, a backing layer may be disposed on the mat.

Preferably, in the dry cream or topical delivery system as described herein, the polymer comprises a synthetic polymer and/or a natural polymer.

Preferably, in the dry cream or topical delivery system as described herein, the polymer is a synthetic polymer, optionally selected from poly(N-isopropylacrylamide) (PNIPAM), polyacrylamide (PAM), poly(2-oxazoline), polyethylenimine (PEI), poly(acrylic acid), polymethacrylate (PMA) and other acrylic polymers, poly(ethylene glycol) (PEG), poly(ethylene oxide) (PEO), poly(vinyl alcohol) (PVA) and copolymers, poly(vinylpyrrolidone) (PVP) and copolymers, polyelectrolytes, cucurbit[n]uril hydrate, HPMC, HPMCAS, and combinations thereof.

Preferably, in the dry cream or topical delivery system as described herein, the polymer is a natural polymer, optionally selected from proteins and carbohydrates; said proteins optionally selected from collagen, gelatin, elastin, silk fibroin, zein, Aloe vera, soy proteins, sodium alginate (SA); said carbohydrates optionally selected from collagen, hyaluronic acid, chitosan, alginates, heparin, pectin, cellulose-based derivatives, starch and modified starch, xylan, xanthan gum and other gums.

Preferably, in the dry cream or topical delivery system as described herein, the polymer is a mixture of polymers.

Preferably, in the dry cream or topical delivery system as described herein, pores in said mat swell upon liquid ingress such that the pores swell by a factor of at least 1.5, 2, 3, 4, 5, 6, 7, 8, 9, 10, 12, 15, 20, 25, 30, 40, 50 or more than 50.

Preferably, in the dry cream or topical delivery system as described herein, said fibers have an average thickness of around 20 nm to 100 μm, around 20 nm to 50 μm, around 20 nm to 20 μm, around 20 nm to 15 μm, around 20 nm to 10 μm, around 50 nm to 10 μm, around 100 nm to 10 μm, around 100 nm to 5 μm, around 150 nm to 5 μm, around 150 nm to 3 μm, around 150 nm to 2 μm, around 150 nm to 1 μm, around 150 nm to 900 nm, around 150 nm to 800 nm, around 150 nm to 700 nm, around 100 nm to 600 nm, around 150 nm to 400 nm, around 200 nm to 400 nm, around 250 nm to around 350 nm; around 50 nm, around 100 nm, around 150 nm, around 200 nm, around 250 nm, around 300 nm or around 350 nm.

Preferably, in the dry cream or topical delivery system as described herein, said fibers are aligned in said mat.

Preferably, in the dry cream or topical delivery system as described herein, said fibres are substantially smooth.

Preferably, in the dry cream or topical delivery system as described herein, said mat has a mean pore size of about 0.1 μm to about 2 μm, about 0.2 μm to about 1.5 μm, about 0.3 μm to about 1.2 μm, about 0.4 μm to about 1 μm, about 0.5 μm to about 0.9 μm, about 0.1 μm to about 1 μm, about 0.2 μm to about 1 μm, about 0.3 μm to about 1 μm, about 0.4 μm to about 1 μm, or about 0.5 μm to about 1 μm.

Preferably, in the dry cream or topical delivery system as described herein, said mat has a total pore volume (TPV) of about 0.1 cc/g or above, about 0.2 cc/g or above, about 0.3 cc/g or above, about 0.4 cc/g or above, about 0.5 cc/g or above, or about 0.6 cc/g or above.

Preferably, in the topical delivery system as described herein, said mat has a stretch force when dry of at least 50 g, at least 100 g, at least 200 g, at least 300 g, at least 400 g, at least 500 g, at least 600 g, at least 700 g, at least 800 g or at least 900 g; and/or
a stretch force in olive oil of at least 50 g, at least 100 g, at least 200 g, at least 300 g, at least 400 g, at least 500 g, at least 600 g, at least 700 g, at least 800 g or at least 900 g; and or
a stretch force in water of at least 50 g, at least 100 g, at least 200 g, at least 300 g, at least 400 g, at least 500 g, at least 600 g, at least 700 g, at least 800 g or at least 900 g.

Preferably, in the topical delivery system as described herein, said mat has a punch force when dry of at least 100 g, at least 200 g, at least 300 g, at least 400 g, at least 500 g, at least 600 g, at least 700 g, at least 800 g, at least 900 g, at least 1000 g, at least 1100 g, at least 1200 g, at least 1300 g, at least 1400 g, at least 1500 g or at least 1600 g; and/or
a punch force in water of at least 100 g, at least 200 g, at least 300 g, at least 400 g, at least 500 g, at least 600 g, at least 700 g, at least 800 g, at least 900 g, at least 1000 g, at least 1100 g, at least 1200 g, at least 1300 g, at least 1400 g, at least 1500 g or at least 1600 g; and/or
a punch force in olive oil of at least 100 g, at least 200 g, at least 300 g, at least 400 g, at least 500 g, at least 600 g, at least 700 g, at least 800 g, at least 900 g, at least 1000 g, at least 1100 g, at least 1200 g, at least 1300 g, at least 1400 g, at least 1500 g or at least 1600 g.

Preferably, in the topical delivery system as described herein, said mat has a tensile strength when dry (initial force (N) required to break the fibers) of at least 1N, at least 2N, at least 3N, at least 4N, at least 5N, at least 6N, at least 7N, at least 8N, at least 9N or at least 10N; and/or
a tensile strength in water (initial force (N) required to break the fibers) of at least 1N, at least 2N, at least 3N, at least 4N, at least 5N, at least 6N, at least 7N, at least 8N, at least 9N or at least 10N; and/or
a tensile strength in olive oil (initial force (N) required to break the fibers) of at least 1N, at least 2N, at least 3N, at least 4N, at least 5N, at least 6N, at least 7N, at least 8N, at least 9N or at least 10N.

Preferably, in the topical delivery system as described herein, said mat can absorb at least ×10, ×15, ×20, ×25, ×30, ×35, ×40, ×45, ×50, ×55, ×60, ×65, ×70, ×75, ×80, ×85, ×90, ×95 or ×100 the mass of non-polar solvent vs the dry mass of the mat.

Preferably, in the topical delivery system as described herein, the polymer is a high molecular weight (HMW) polymer.

Preferably, in the topical delivery system as described herein, the polymer has a weight average molecular weight of 300 KDa or above, 320 KDa or above, 340 KDa or above, 360 KDa or above, 380 KDa or above, 400 KDa or above, 420 KDa or above, 440 KDa or above, 460 KDa or above, 480 KDa or above, 500 KDa or above, 520 KDa or above, 540 KDa or above, 560 KDa or above, 580 KDa or above, 600 KDa or above, 620 KDa or above, 640 KDa or above, 660 KDa or above, 680 KDa or above, or 700 KDa or above.

Preferably, in the dry cream or topical delivery system as described herein, said mat is wet with a solvent, cream, foam, gel, lotion or ointment.

Preferably, in the dry cream or topical delivery system as described herein, said mat is wet with a non-polar medium or a polar medium.

Preferably, in the topical delivery system as described herein, said bioactive agent(s) are attached to the surface of said fibres; said bioactive agent(s) are in the form of fibres and said bioactive fibres and said hydrophilic polymeric fibers are formed together; said bioactive agent(s) are integrated into said hydrophilic polymeric fibers; said hydrophilic polymeric fibers are tubular and said bioactive agent(s) is encased within said fibres; said hydrophilic polymeric fibers form a scaffold that incorporates or retains a solution comprising or consisting of the bioactive agent(s).

Preferably, in the topical delivery system as described herein, said bioactive agents are selected from essential oils, antioxidants, vitamins, peptides, or combinations thereof.

Preferably, in the topical delivery system as described herein, the fibers further incorporate one or more surfactant(s).

Preferably, in the topical delivery system as described herein, further particulates are suspended a liquid medium wetting said mat.

Preferably, in the dry cream or topical delivery system as described herein, said topical delivery system is for delivery to the skin or to the mucous membranes.

Preferably, in the topical delivery system as described herein, said topical delivery system is a mask, optionally a facemask.

Preferably, in the topical delivery system as described herein, said topical delivery system comprises a backing layer disposed on said mat.

In a further aspect of the present invention there is provided a wet cream produced by mixing a dry cream as described herein with a wetting agent.

In a further aspect of the present invention there is provided a cream or gel produced by mechanical agitation of a topical delivery system as described herein with a wetting agent.

In a further aspect of the present invention there is provided a process of manufacturing a dry cream as described herein, comprising a step of electrospinning a polymer with oil(s).

In a further aspect of the present invention there is provided a process of manufacturing a dry cream as described herein, comprising a step of electrospinning a polymer with a bioactive agent(s).

In a further aspect of the present invention there is provided a method of topical delivery comprising applying a dry cream or topical delivery system as described herein to the user.

Preferably, said mat is applied to the user after wetting, or said mat is applied to the user after wetting.

Preferably, said mat is wetted with a non-polar medium, for example oil; a polar medium, for example water; or an emulsion medium.

Preferably, said medium is a solvent, cream, foam, gel, lotion or ointment.

Preferably, said mat is applied to the user for at least 10 minutes, at least 15 minutes, at least 20 minutes, at least 25 minutes or at least 30 minutes.

The dry cream or topical delivery system may be used for cosmetic or therapeutic purposes.

In a further aspect, there is provided a dry cream or topical delivery system as defined herein, for use as a medicament.

In a further aspect, there is provided use of a dry cream or topical delivery system as defined herein, in the manufacture of a medicament.

In a further aspect, there is provided a method of treatment comprising using a dry cream or topical delivery system as defined herein.

In a further aspect, there is provided a use of a dry cream or topical delivery system as defined herein for cosmetic purposes.

In a further aspect, there is provided a cosmetic method comprising applying a dry cream or topical delivery system as defined herein to a user.

The following features apply to all aspects of the invention.

### BRIEF DESCRIPTION OF THE FIGURES

FIG. 1a shows a diagram illustrating the different parts of a Franz cell.

FIG. 1b shows a fiber mat made according to the present invention.

FIG. 2 shows SEM images of F_{PEO1} (a), F_{PEO2} (b) and F_{PEO3} (c).

FIG. 3 shows SEM image of F_{PS2} (a) and pore size measured with ImageJ (b).

FIG. 4 shows pores size distribution obtained using ImageJ.

FIG. 5 shows a digital photograph of the F_{PS2} mat (a) inside the simulated sweat, (b) after removal from media; (c) after rubbing the F_{PS2} mat.

FIG. 6 shows a tensile stress-strain curve of the F_{PS} mat.

FIG. 7 shows a tensile stress-strain curve of the F_{PEO3} (top line) and F_{PEO2} (bottom line) mats.

FIG. 8 shows caffeine permeation from 2% caffeine solution, F_{PVP} and F_{PEO} soaked in the same caffeine solution.

FIG. 9 shows caffeine permeation from 1% caffeine solution, FPMA and F_{PEO} soaked in the same caffeine solution.

FIG. 10 shows reversing signals of the MTDSC results of each individual polymer (A=HPMCAS and B=PVP), the polymer blend (C=HPMCAS-PVP) and the drug loaded polymer matt (D=HPMCAS-PVP-MET).

FIG. 11 shows SEM images of (A) HPMCAS-PVP 1:1 blend fiber matts, (B) PVP fiber matts and (C) PEO Fiber matts at 3,500× magnification.

FIG. 12 shows fluorescent images of polymer mats: A=HPMCAS-PVP with no oil added B=HPMCAS-PVP with oil added C=PVP 10% with no oil added D=PVP 10% with oil added.

FIG. 13 shows fluorescent images of A=HPMCAS-PVP without oil B=HPMCAS-PVP without oil with a hole C=HPMCAS-PVP with oil D=HPMCAS-PVP with oil with a hole.

FIG. 14 shows comparison of the amount of force required to break the polymer matts with and without oil. (A) PVP—AUC dry=1.961 J, AUC with oil=3.722 J; (B) PEO—AUC dry=25.008 J, AUC with oil=41.052 J; (C) HPMCAS-PVP—AUC dry=1.216 J, AUC with oil=0.804 J.

FIG. 15 shows SEM images of the bilayer mask at a magnification of (A) 1000×; (B) 5000×; and (C) 10000×. Arrows indicate zein layer.

FIG. 16 shows SEM images of the bilayer mask after it has undergone wetting, then being dried at room temperature, at a magnification of (A) 1000×; (B) 5000×; and (C) 10000×. Arrows indicate particles of SA/PEO formed from the inner mask layer.

FIG. 17 shows photographs of (A) a PVA/PVP dry cream formulation after wetting; and (B) a gelatin dry cream formulation after wetting.

FIG. 18 shows burst strength testing of an electrospun tea tree oil-based PVA/PEO dry cream (testing in triplicate, n=3).

FIG. 19 shows burst strength testing of an electrospun turmeric oil-based PVA/PEO dry cream (testing in triplicate, n=3).

FIG. 20 shows: (A) a scanning electron microscopy image of an electrospun tea tree oil-based PVA/PEO dry cream—magnification power 3,000×; average fiber diameter, 376 nm±108 nm, n=100. (B) a scanning electron microscopy image of an electrospun turmeric oil-based PVA/PEO dry cream—magnification power 3,000×; average fiber diameter, 559 nm±216 nm, n=100. (C) a scanning electron microscopy image of an electrospun castor oil-based PVA/PVP dry cream (Example 11-1), magnification power 3,000×; the fiber morphology was beaded fiber. (D) an optical microscopy image of an electrospun castor oil-based pectin/PEO dry cream, magnification power 3,000×.

FIG. 21 shows a water content analysis diagram for an electrospun tea tree oil-based PVA/PEO dry cream.

FIG. 22 shows a water content analysis diagram for an electrospun turmeric oil-based PVA/PEO dry cream.

### DETAILED DESCRIPTION OF THE INVENTION

In the present application, a number of general terms and phrases are used, which should be interpreted as follows.

The present invention has developed novel, topical delivery methods which have superior sensation and appearance qualities to current products, can significantly increase treatment effectiveness, and which reduce the manufacturing cost of topical products. The present invention allows for high flexibility in terms of the active ingredients that can be included.

By “wetted” in relation to the dry cream and the topical delivery system as described herein, it is meant that the mat has sufficient wetting agent, particularly water, so that the mat is wet. “Wetted” may refer to where the mat and wetting agent are combined in w/w ratio of about 1:1 to 1:50, about 1:2 to about 1:40, about 1:3 to about 1:30, about 1:4 to about 1:25, about 1:5 to about 1:20, about 1:6 to about 1:19, about 1:7 to about 1:18, about 1:8 to about 1:17, about 1:9 to about 1:16, or about 1:10 to about 1:15.

By “wetting agent” in relation to the dry cream and the topical delivery system as described herein, this may refer to a medium comprising a non-polar medium and/or a polar medium that may be applied to cause wetting. In some embodiments, the polar medium is an aqueous medium (e.g. water).

By “mat” it is meant a collection of fibers formed into a desired shape to be applied to the user. The mat can form a substrate upon which a bioactive agent or agents can be deposited. The mat may either be formed incorporating bioactive agent(s) or it may be formed without and the bioactive agent(s) added through the addition of a solvent containing said bioactive agent(s). The mat may be considered a membrane.

The mat is formed by electrospinning. Electrospinning is a facile technique which permits the production of functional nanomaterials from mixed solution of a polymer and a functional component. The polymeric solution is expelled at a controlled rate from a needle, and a high potential difference applied between the needle and a grounded collector. The voltage causes rapid evaporation of the solvent, resulting in nano- to microscale fibers with diameters of a reproducible range.

By “hydrophilic” in the context of a polymer, it is meant that the contact angle a droplet of water makes with the surface of the polymer is less than 90°. Conversely, a “hydrophobic” polymer refers to where the contact angle a droplet of water makes with the surface of the polymer is more than or equal to 90°. For example, the contact angle may be less than 80°, preferably less than 70°, more preferably less than 60°, even more preferably less than 50°, yet even more preferably less than 40°. In some embodiments, the contact angle may be about 5° to about 80°, preferably about 100 to about 70°, more preferably about 150 to about 60°, even more preferably about 200 to about 50°, yet even more preferably about 250 to about 400.

### Dry Cream

In a first embodiment, there is provided a dry cream for delivery of oil(s) to a user, said dry cream comprising a mat of electrospun hydrophilic polymeric fibers containing said oil(s).

By “dry cream” it refers to a material comprising a dried mat of fibers that can be reactivated by mixing the mat with a wetting agent to form a “wet” version of the cream. Dry creams according to the present invention lack a backing layer. Dry creams according to the present invention are substantially free of water.

The term “dry” as used in “dry cream” may refer to a material that comprises less than 2.0 wt % of water, less than 1.9 wt % of water, less than 1.8 wt % of water, less than 1.7 wt % of water, less than 1.6 wt % of water, less than 1.5 wt % of water, less than 1.4 wt % of water, less than 1.3 wt % of water, less than 1.2 wt % of water, less than 1.1 wt % of water, less than 1.0 wt % of water, less than 0.9 wt % of water, less than 0.8 wt % of water, less than 0.7 wt % of water, less than 0.6 wt % of water, less than 0.5 wt % of water, less than 0.4 wt % of water, less than 0.3 wt % of water, less than 0.2 wt % of water, less than 0.1 wt % of water, less than 0.09 wt % of water, less than 0.08 wt % of water, less than 0.07 wt % of water, less than 0.06 wt % of water, less than 0.05 wt % of water, less than 0.04 wt % of water, less than 0.03 wt % of water, less than 0.02 wt % of water, or less than 0.01 wt % of water, based on a total weight of the dry cream.

The fibers of the dry cream may dissolve in the wetting agent, thereby forming an emulsion. The emulsion may comprise an aqueous liquid phase comprising the dissolved fibers and an oil phase comprising said oil(s).

The mat of the dry cream may comprise any suitable biocompatible hydrophilic or polar polymer or polymer blends. Suitable polymers include synthetic and natural polymers.

Suitable synthetic polymers include (but are not limited to): poly(N-isopropylacrylamide) (PNIPAM), polyacrylamide (PAM), poly(2-oxazoline), polyethylenimine (PEI), poly(acrylic acid), polymethacrylate (PMA) and other acrylic polymers, poly(ethylene glycol) (PEG), poly(ethylene oxide) (PEO), poly(vinyl alcohol) (PVA) and copolymers, poly(vinylpyrrolidone) (PVP) and copolymers, polyelectrolytes, cucurbit[n]uril hydrate, HPMC, HPMCAS and the like. In a preferred embodiment, the synthetic polymer may be selected from PVA, PVP, PEO and HPMC or combinations thereof. In a further preferred embodiment, the synthetic polymer may be selected from PVA, PVP and HPMC or combinations thereof. In an even further preferred embodiment, the synthetic polymer may comprise a combination of PVA with PVP, PVA with HPMC, or PVA with PEO; preferably PVA with PEO. The polymers may include a wide range of substituent groups.

Suitable natural polymers include proteins and carbohydrates.

Suitable proteins include (but are not limited to): collagen, gelatin, elastin, silk fibroin, zein, Aloe vera, soy proteins, sodium alginate (SA), and the like. In a preferred embodiment, the natural polymer may be gelatin.

Suitable carbohydrates include (but are not limited to): hyaluronic acid (with a range of molecular weight), chitosan, alginates, heparin, pectin, cellulose-based derivatives, starch and modified starch, xylan, xanthan gum and other gums. In a preferred embodiment, the natural polymer may be pectin (for example, in combination with a synthetic polymer such as PEO).

The mat can comprise combinations of polymers.

The mat of the dry cream may comprise a mixture of synthetic and natural polymers as described above.

The mats of the present invention are preferably biodegradable whereby the polymer strands are susceptible to hydrolysis. Alternatively, the polymers may be enzymatically degradable.

The total polymeric fiber content may be about 40 wt % to about 99 wt %, about 42 wt % to about 98 wt %, about 44 wt % to about 97 wt %, about 46 wt % to about 96.5 wt %, about 48 wt % to about 96.5 wt %, or about 50 wt % to about 96.5 wt %, based on a total weight of the dry cream.

The oil(s) used in the dry cream are not particularly limited. The term “oil” may refer to a liquid substance (at room temperature (20° C.) and pressure) that is immiscible with water. Non-limiting examples of oil(s) include castor oil, rapeseed oil, soybean oil, sunflower oil, olive oil, Camellia oil, mustard oil, fish oil, Labrafac PG; essential oils such as lemon oil, lemongrass oil, grapefruit oil, basil oil, sage oil, clove oil, tea tree oil, aniseed oil, bay oil, bergamot oil, birch oil, black pepper oil, black spruce oil, camphor white oil, caraway seed oil, cardamom oil, carrot seed oil, Cassia oil, cedarwood oil, cinnamon oil, Citronella java oil, clary sage oil, clove bud oil, cypress oil, Eucalyptus oil, fir needle oil, frangipani oil, frankincense, geranium oil, ginger oil, jasmine oil, lavender oil, lime oil, lotus pink oil, lotus white oil, marjoram oil, orange oil, osmanthus oil, patchouli oil, peppermint oil, rosemary oil, rose oil, rose morocco oil, rose otto oil, rosewood oil, sage Dalmatian oil, sandalwood oil, spearmint oil, tangerine oil, tuberose oil, turmeric oil, wintergreen oil, and Ylang Ylang oil.

The total oil content of the dry cream may be about 1 wt % to about 35 wt %, about 1 wt % to about 30 wt %, about 1 wt % to about 25 wt %, about 1 wt % to about 20 wt %, about 1 wt % to about 15 wt %, about 1 wt % to about 10 wt %, about 2 wt % to about 8 wt %, about 3 wt % to about 6 wt %, or about 3.5 wt % to about 5 wt %, based on a total weight of the dry cream.

A w/w ratio of the total oil content to the total polymeric fiber content may be from about 1:5 to about 1:50, about 1:6 to about 1:45, about 1:7 to about 1:40, about 1:8 to about 1:35, about 1:9 to about 1:30, or about 1:10 to about 1:25.

The oil(s) may be encased within the fibers. In further embodiments, the oil(s) may be additionally coated (in addition to being encased) on the fibers and/or located within pores between fibers.

The dry cream may comprise one or more surfactant(s). Non-limiting examples of surfactants include non-ionic surfactants, such as polyoxylglycerides (e.g. Labrasol, Labrafil M 1944 CS), polyoxylethylene stearates (e.g. Tefose 63) and poloxamers (e.g. Pluronic F-127).

The total surfactant content may be about 20 wt % to about 70 wt %, about 25 wt % to about 65 wt %, about 30 wt % to about 60 wt %, about 35 wt % to about 55 wt %, or about 40 wt % to about 50 wt %.

A w/w ratio of the total polymeric fiber content to the total surfactant content may be from about 1:5 to about 1:0.5, about 1:4 to about 1:0.6, about 1:3 to about 1:0.7, about 1:2 to about 1:0.8, or about 1:1 to about 1:0.9.

A w/w ratio of the total oil content to the total surfactant content may be from about 1:2 to about 1:20, about 1:3 to about 1:18, about 1:4 to about 1:16, about 1:5 to about 1:14, about 1:6 to about 1:12, about 1:7 to about 1:10, or about 1:8 to about 1:9.

The dry cream may comprise bioactive agent(s) as described herein. The dry cream may be for delivery of oil(s) and bioactive agent(s) to a user.

The dry cream may be applied directly to the user, mixed with a wetting agent, then the “wet” version of the cream applied to the user. Alternatively, the dry cream may be mixed with a wetting agent prior to application to the user, then the “wet” version of the cream applied to the user.

The dry cream as described herein may be manufactured by using a process comprising a step of electrospinning a polymer with oil(s). The polymer may be as described herein. The oil may be as described herein. Other additives (e.g. surfactants, bioactive agent(s)) may also be used during the process of manufacture.

The dry cream may be packaged as part of a kit comprising the dry cream. The kit may further comprise instructions to mix the dry cream with a wetting agent to produce a “wet” version of the cream.

### Topical Delivery Systems

In a second embodiment, there is provided a topical delivery system for delivery of bioactive agent(s) to a user, said topical delivery system comprising a mat of electrospun hydrophilic polymeric fibers containing said bioactive agent(s), wherein said mat is configured to substantially retain its shape on a mounting surface when exposed to a wetting agent but disintegrate under mechanical agitation to form a cream or gel.

By “topical delivery system” it is meant a system which can be applied to a user so as to topically deliver one or more bioactive agents. Delivery may be to the skin, mucous membrane or the like. If delivered to the face of a user, for example in the context of a cosmetic, the topical delivery system may be a mask. In a preferred embodiment, the topical delivery system is a facemask.

The topical delivery system has particular properties. It is able to substantially retain its shape on a mounting surface while being applied by the user. This allows the system to be applied by the user to the target area. The system can be wetted and still substantially retains its shape on the mounting surface. This is in contrast with prior art systems (for example masks) which dissolve on contact with water.

By “substantially retain its shape on a mounting surface” it is meant that the mat will not dissolve upon application of a wetting agent on the mounting surface, for example when the mat is exposed to an aqueous medium (e.g. water). In some cases, the polymer fibers of the mat may remain intact on the mounting surface when exposed to the wetting agent and the overall structure is maintained by a network formed by the polymer fibers. In other cases, the polymer fibers may form solid polymer particles when exposed to the wetting agent on the mounting surface but the overall structure of the mat may nevertheless be maintained by dynamically formed crosslinks (e.g. ionic bonds and/or non-covalent interactions such as hydrogen bonds) formed between the solid polymer particles. The polymer fibers or particles can thereby can become suspended in a wetting agent to form a heterogeneous mixture comprising a solid phase (e.g. particles) and a liquid phase (e.g. wetting agent), but still held together on the mounting surface. In this circumstance, the mat will not disintegrate. The mat may not disintegrate at room temperature (e.g. about 20-25° C.) or at body temperature (e.g. about 35-40° C.).

By “disintegrate” it is meant that the overall structure of the mat is disrupted such that portions of the solid phase (e.g. fibers or particles) become separated away from other portions of the solid phase (e.g. fibers or particles) by breaking interactions (e.g. ionic bonds and/or non-covalent interactions such as hydrogen bonds) between the various portions of the solid phase. Disintegration is distinguished from dissolution as the solid phase is not substantially solubilized by the liquid phase to form a homogeneous phase.

In some cases, the wetted mat may be directly applied by the user to the body using the mounting surface without it disintegrating or dissolving, for example as a mask (e.g. facemask). The mounting surface is not particularly limited and represents any solid surface the user may use in order to transfer the wetted mat onto a body. For example, the user could wet the topical delivery system on their hand (such that the hand itself acts as a mounting surface), and then use their hand to apply the wetted mat to the body. In another example, the mat may be pre-supplied with a backing layer, allowing the user to lift and apply the wetted mat to the body. In other words, the mat does not disintegrate simply by being wetted on the mounting surface. During this period, the mat may topically deliver bioactive agent(s) to the user. The mats of the present invention may not disintegrate unless they are subjected to mechanical agitation.

Where particles are generated during wetting, the particles may have various sizes. For example, the particles may have an average particle diameter of around 20 nm to 100 μm, around 20 nm to 50 μm, around 20 nm to 20 μm, around 20 nm to 15 μm, around 20 nm to 10 μm, around 50 nm to 10 μm, around 100 nm to 10 μm, around 100 nm to 5 μm, around 150 nm to 5 μm, around 150 nm to 3 μm, around 150 nm to 2 μm, around 150 nm to 1 μm, around 150 nm to 900 nm, around 150 nm to 800 nm, around 150 nm to 700 nm, around 100 nm to 600 nm, around 150 nm to 400 nm, around 200 nm to 400 nm, around 250 nm to around 350 nm; around 50 nm, around 100 nm, around 150 nm, around 200 nm, around 250 nm, around 300 nm or around 350 nm.

By “mechanical agitation” it is meant that the user physically rubs or otherwise disturbs the mat structure, for example by subjecting the mat structure to a shear stress. Upon mechanical agitation the polymer fibers disintegrate such that the mat substantially turns into a cream or gel like consistency. From a user perspective, the disintegrated mat will have a cream or gel like sensation.

“Cream or gel like” is intended to convey that the topical delivery device converts from a mat like fiber structure into a physical state that loses identity of the individual fibers and has a cream or gel consistency. The disintegrated mat may be substantially uniformly cream or gel like after mechanical agitation.

By “cream” or “gel” for the topical delivery system, it is intended to refer to a material that comprises a solid phase (e.g. fibers or particles as described above) expanded throughout its volume by a liquid phase. The liquid phase may comprise an aqueous liquid phase and an oily liquid phase.

The mat only substantially disintegrates under mechanical agitation. This contrasts with mats that dissolve in the presence of an aqueous solution, for example when applied to a wet face or when wetted on a face. The mats of the present invention retain their structure on the mounting surface under these conditions and only disintegrate when mechanically agitated.

The mat may remain substantially in shape on the mounting surface for at least 3 minutes when wetted, at least 5 minutes when wetted, at least 10 minutes when wetted, at least 20 minutes when wetted, at least 30 minutes when wetted, at least 60 minutes when wetted, or will substantially remain in shape on the mounting surface while wetted.

The mat may comprise any suitable biocompatible hydrophilic or polar polymer or polymer blends. Suitable polymers include synthetic and natural polymers.

Suitable synthetic polymers include (but are not limited to): poly(N-isopropylacrylamide) (PNIPAM), polyacrylamide (PAM), poly(2-oxazoline), polyethylenimine (PEI), poly(acrylic acid), polymethacrylate (PMA) and other acrylic polymers, poly(ethylene glycol) (PEG), poly(ethylene oxide) (PEO), poly(vinyl alcohol) (PVA) and copolymers, poly(vinylpyrrolidone) (PVP) and copolymers, polyelectrolytes, cucurbit[n]uril hydrate, HPMC, HPMCAS and the like. In a preferred embodiment, the synthetic polymer may be selected from PVP, PEO and HPMCAS, or combinations thereof. The polymers may include a wide range of substituent groups.

Suitable natural polymers include proteins and carbohydrates.

Suitable proteins include (but are not limited to): collagen, gelatin, elastin, silk fibroin, zein, Aloe vera, soy proteins, sodium alginate (SA), and the like. In a preferred embodiment, the natural polymer may be sodium alginate.

Suitable carbohydrates include (but are not limited to): hyaluronic acid (with a range of molecular weight), chitosan, alginates, heparin, pectin, cellulose-based derivatives, starch and modified starch, xylan, xanthan gum and other gums.

The mat may comprise a mixture of synthetic and natural polymers as described above. In a preferred embodiment, the mat comprises PEO/SA.

The mat can comprise combinations of polymers.

The mats of the present invention are preferably biodegradable whereby the polymer strands are susceptible to hydrolysis. Alternatively, the polymers may be enzymatically degradable.

The mats of the present invention may further comprise a backing layer disposed on the mat.

In some embodiments, the backing layer may be configured to substantially retain its shape when exposed to a wetting agent but disintegrate under mechanical agitation to form a cream or gel.

The backing layer may comprise electrospun natural polymers as described herein. The natural polymer may contain hydrophilic functional groups within the molecular structure that allow the material to be wetted after in contact with water, thereby providing water sorption capabilities. The backing layer may have a porous structure, thereby providing oil sorption capabilities. Non-limiting examples of materials that can be used for the backing layer include proteins, polysaccharides and carbohydrates, for example pea protein, cross-linked alginate, chitosan and zein. In a preferred embodiment, the backing layer comprises zein.

The backing layer may be useful for helping the mats of the present invention to be lifted and applied to a user without it disintegrating or dissolving, for example as a mask (e.g. facemask). The backing layer may be useful for mats that are configured to form solid particles when exposed to the wetting agent but where the overall structure of the mat is nevertheless be maintained by dynamically formed crosslinks formed between the solid particles.

Selecting the appropriate polymer enables tailoring of the properties of the topical delivery system.

The suitability of a polymer for electrospinning is dependent on its viscosity, conductivity and surface tension. The skilled person will select a suitable polymer that can be processed for electrospinning.

Provided the polymer is suitable for electrospinning, its properties will be selected based on its properties in use. Suitable polymers will have sufficient strength when wetted (for example in aqueous solution) so that the mat substantially retains its shape. The polymer can also disintegrate under mechanical agitation.

Preferably, this may be achieved through selecting polymers with an appropriately high weight average molecular weight (Mw). The weight average molecular weight may be 300 KDa or above, 320 KDa or above, 340 KDa or above, 360 KDa or above, 380 KDa or above, 400 KDa or above, 420 KDa or above, 440 KDa or above, 460 KDa or above, 480 KDa or above, 500 KDa or above, 520 KDa or above, 540 KDa or above, 560 KDa or above, 580 KDa or above, 600 KDa or above, 620 KDa or above, 640 KDa or above, 660 KDa or above, 680 KDa or above, or 700 KDa or above. The skilled person will select a suitable molecular weight for the polymer in question to enable the electrospun mat to substantially retain its shape when wetted but disintegrate under mechanical agitation.

In an embodiment, the mats have a stretch force of at least 50 g, at least 100 g, at least 200 g, at least 300 g, at least 400 g, at least 500 g, at least 600 g, at least 700 g, at least 800 g or at least 900 g. In an embodiment, this is when the mat is dry. In a further embodiment, this is when the mat is wetted in water. In a further embodiment, this is when the mat is wetted in olive oil.

In an embodiment, the mats have a punch force of at least 100 g, at least 200 g, at least 300 g, at least 400 g, at least 500 g, at least 600 g, at least 700 g, at least 800 g, at least 900 g, at least 1000 g, at least 1100 g, at least 1200 g, at least 1300 g, at least 1400 g. at least 1500 g or at least 1600 g. In an embodiment, this is when the mat is dry. In a further embodiment, this is when the mat is wetted in water. In a further embodiment, this is when the mat is wetted in olive oil.

In an embodiment, the dry mats have a tensile strength (initial force (N) required to break the fibers) of at least 1N, at least 2N, at least 3N, at least 4N, at least 5N, at least 6N, at least 7N, at least 8N, at least 9N or at least 10N.

In a further embodiment, the oil soaked mats have a tensile strength (initial force (N) required to break the fibers) of at least 1N, at least 2N, at least 3N, at least 4N, at least 5N, at least 6N, at least 7N, at least 8N, at least 9N or at least 10N.

In a further embodiment, the water soaked mats have a tensile strength (initial force (N) required to break the fibers) of at least 1N, at least 2N, at least 3N, at least 4N, at least 5N, at least 6N, at least 7N, at least 8N, at least 9N or at least 10N.

The mats according to the present invention are strong whilst dry and maintain sufficient shape when wet. In some case, mats are stronger when soaked in oil. This is beneficial, as delicate fibers mats may lead to high waste during handling by either the manufacturer or the consumer.

In a further embodiment, the mats can absorb at least ×10 the mass of non-polar solvent vs the dry mass of the mat. Preferably the mats can absorb at least ×15, ×20, ×25, ×30, ×35, ×40, ×45, ×50, ×55, ×60, ×65, ×70, ×75, ×80, ×85, ×90, ×95 or ×100 the mass of non-polar solvent vs the dry mass of the mat.

### Other Characteristics, Methods of Production and Uses of the Dry Creams and Topical Delivery Systems

Preferably the polymer will swell upon contact with the delivery solvent system.

It is noted that although the polymers are hydrophilic, they allow for the uptake of non-polar mediums. This is believed to be due to capillary action which allows for a non-polar substance to be incorporated into the voids of polar-fibers.

The present invention may further incorporate additives. Suitable additives include (but are not limited to) non-polar additives such as vitamin E, castor oil and peppermint oil. Additives may include surfactants to form an emulsion that facilitates electrospinning. Examples of suitable surfactants include non-ionic surfactants such polyoxylglycerides (e.g. Labrasol, Labrafil M 1944 CS), polyoxylethylene stearates (e.g. Tefose 63) and poloxamers (e.g. Pluronic F-127).

The electrospinning parameters can be modified to adjust the properties of the fiber mats. Any suitable electrospinning technique is encompassed by the present application, including single nozzle electrospinning, co-axial electrospinning, needle or free surface electrospinning, melt electrospinning or melt blowing (see, for example Brown et al, Adv Mater. 2011 Dec. 15; 23(47):5651-7. doi: 10.1002/adma.201103482).

In an embodiment, mat uptake properties are modified by adjusting electrospinning parameters. In an embodiment, increasing electrospinning voltage can modify fiber properties. Voltages may be greater than 10 kV, greater than 15 kV or greater than 20 kV. Increasing voltage may increase mechanical strength of the mat fibers.

In an embodiment, the mats according to the present invention can hold a higher volume of liquid than would be predicted from their total pore volume (TPV). This is believed to be due swelling of the pores during liquid uptake. In an embodiment, the mat swells by a factor of at least 1.5, 2, 3, 4, 5, 6, 7, 8, 9, 10, 12, 15, 20, 25, 30, 40, 50 or more than 50.

Oil sorption capacity can be affected by various properties of the fibers (diameter, surface area, lipophilicity and density) and the oil (viscosity) as well as the interaction between the oil and the polymer mats. Having large inter-fiber spaces can increase the storage volume of the oil; though these spaces may not be excessively large leading to oil drainage.

In an embodiment, the fibers have an average thickness of around 20 nm to 100 μm, around 20 nm to 50 μm, around 20 nm to 20 μm, around 20 nm to 15 μm, around 20 nm to 10 μm, around 50 nm to 10 μm, around 100 nm to 10 μm, around 100 nm to 5 μm, around 150 nm to 5 μm, around 150 nm to 3 μm, around 150 nm to 2 μm, around 150 nm to 1 μm, around 150 nm to 900 nm, around 150 nm to 800 nm, around 150 nm to 700 nm, around 100 nm to 600 nm, around 150 nm to 400 nm, around 200 nm to 400 nm, around 250 nm to around 350 nm; around 50 nm, around 100 nm, around 150 nm, around 200 nm, around 250 nm, around 300 nm or around 350 nm. The molecular weight of the polymer, its surface tension and evaporation rate may influence polymer diameter.

In an embodiment, the mat fibers are aligned. This may have the effect of increasing tensile strength. This may also have the effect of reducing the total uptake volume. The appropriate degree of alignment will depend on need. Fibers may be aligned by the collection method with a rotating collector achieving greater alignment whereas a static collector creates mats with non-aligned fibers, for example a non-woven mat.

In an embodiment, the fibers are smooth and are substantially straight. By substantially straight it is meant that individual fibers have a degree of straightness when visually inspected on a microscope. In a further embodiment, the fibers have wrinkles.

In an embodiment, the mats and/or the backing layer (when present) according to the present invention have a mean pore size of about 0.1 μm to about 2 μm, about 0.2 μm to about 1.5 μm, about 0.3 μm to about 1.2 μm, about 0.4 μm to about 1 μm, about 0.5 μm to about 0.9 μm, about 0.1 μm to about 1 μm, about 0.2 μm to about 1 μm, about 0.3 μm to about 1 μm, about 0.4 μm to about 1 μm, or about 0.5 μm to about 1 μm.

In an embodiment, the mats and/or the backing layer (when present) according to the present invention have a total pore volume (TPV) of about 0.1 cc/g or above, about 0.2 cc/g or above, about 0.3 cc/g or above, about 0.4 cc/g or above, about 0.5 cc/g or above, or about 0.6 cc/g or above.

Mean pore size and total pore volume may be measured using a BET method using N₂ as an adsorption gas.

The topical delivery system as described herein may be manufactured by using a process comprising a step of electrospinning a polymer with bioactive agent(s). The polymer may be as described herein. The bioactive agent(s) may be as described herein. Other additives (e.g. surfactants) may also be used during the process of manufacture. The present invention enables the topical delivery of bioactive agents. The bioactive agent can be presented in any form suitable for topical or transdermal delivery.

The bioactive agent(s) can be any active ingredient that may be useful in topical or transdermal application. The bioactive agents can either be an agent that can be solved or suspended in the electrospinning solution. Via this approach, the bioactive agent will be incorporated into the fiber mat.

In an alternative embodiment, the mat is formed and the bioactive agent(s) are subsequently applied.

In an alternative embodiment, the bioactive agent(s) may be provided in a separate solvent which is used to wet the dried fiber mat before use. In this embodiment, the bioactive agent may be incorporated into the form of a include cream, foam, gel, lotion, ointment and the like. A combination of both approaches is possible. One or more than one bioactive agent may be used.

Thus, the bioactive agent and polymer carried can be incorporated together in a variety of ways. 1) Bioactive agent(s) can be attached to the surface of the carrier which is in the form of nanofibers or microfibers; 2) both bioactive agent(s) and carrier are in nanofiber- or microfiber-form thereby having multiple nanofibers or microfibers interlaced together; 3) the bioactive agent(s) and carrier material are integrated into a single fiber; 4) the carrier material is electrospun into a tubular form encasing the bioactive agent(s); 5) the carrier material forms a scaffold which can incorporate or retain a solution comprising or consisting of the bioactive material(s).

Suitable bioactive agent(s) and/or oil(s) include essential oils, antioxidants, vitamins, peptides, hyaluronic acid and hyaluronate salts, enzymes and coenzyme such as Q10, yeast extract, botanic extract, fruit and plant extracts, minerals, collagen, marine extracts, alga, active carbon or combinations thereof. Further bioactive agents may be selected from a vitamin chosen from: vitamin A, enulin, fructans, vitamin B1 (thiamine), vitamin B-2 (riboflavin), vitamin B-3 (niacine), vitamin B-6, vitamin B-5, vitamin B-17 (amygdaline/nitriloside), vitamin C, vitamin D, vitamin F, vitamin K, vitamin P, Alpha-linolenic Acid (ALA), Alpha hydroxyl acid (AHA), Ascorbic Acid, beta-bisabolene, beta carotene, biotin, copper, biotin, calcium, carotenes, carotenoids, chromium, copper, ferulic Acid and foliate, iodine, iron, manganese, magnesium, molybdenum, nicacinamide, nicotinic acid, panthothenic acid, potassium, retinol, selenium, zinc; an vegetal extract chosen from: a botanical extract, Aloe vera extract, Myrrh extract, burdock root extract (0.9% Chlorogenic Acid), Chicory root extract, nettle root extract (1% silica), juice extract, gel extract, plant vinegar extract, fruit vinegar extract, herbal vinegar extract, grain vinegar extract, vegetable vinegar extract, caffeine extract; botanical extract granules; plankton extract; silver extract; Solanum lycopersicum extract; Solidago virgaurea extract; and another suitable supplement chosen from: a botanical extract, rose geranium floral distilled water, Bio-electrolyte Spring Water, Vegetal glycerin, Aloe barbadensis Leaf Juice, Sodium Lactate, Commiphora myrrha, Vitis grape Tannin Extract, Centella asiatica, Gotu Kola extract, Niacinamide, MSM, Methylsulfonylmethane (natural sulfur), Inositol, Sodium Starch Octenyl Succinate, Calcium Pantothenate, Maltodextrin, Sodium Ascorbyl Phosphate, Tocopheryl Acetate, Pyridoxine HCI, Silica, Cichorium intybus, Alpha-glucan oligosaccharide, Gluconolactone, Sodium citrate, Magnesium Chloride mineral, Sodium Benzoate, Dehydroacetic acid (and) Benzyl alcohol, d-panthenol, Arctium lappa, Urtica dioica, Sodium bicarbonate, electrolyte, distilled water, carbonized water, mineralized water, D20 heavy water, deuterium oxide, semi-heavy water, heavy oxygen water, water, and humectants, hydrolats, infusion, decoction, maceration, glycerin (vegetal), corn alcohol, grain alcohol, fruit alcohol, ethanol, honey, acetamine MEA, acetone, acetum, actetyl-1-carnitine, acetyl hexapeptide-3, acetyl pentapeptide-1, acetyl thyrosine, adenosine phosphate, adenosine triphosphate, adipic acid, aescin, agar, agave, algae, algin, alginate, alginic acid, alkaloids, allantoin, Aloe vera, alpha-glucan oligosaccharide, alpha hydroxyl acids (AHA's), alpha-hydroxyoctanoic acid, alpha-isomethyl ionone, alpha lipoic acid, Alteromonas ferment, alum, aluminium distearate, aluminum hydroxide, aluminum lactate, aluminum starch octenylsuccinate, Aluminum stearate, aluminium tristearate, aluminium/magnesium hydroxide stearate, aminopropyl ascorbyl phosphate (AAP), amorphophallus konjac root powder, anthocyanidins, anthquirone, antimicrobial, antibacterial, apigenin, Arabidopsis acid, arbutin, argireline, astringent, aucubin, azelaic acid, Bacillus ferment, beta-glucan, beta hydroxyl acids (BHA's), berberine, biochanin, boric acid, bromelain, botswella, brewers yeast, Caesalpinia spinosa gum, calcium pantothenate, carbomer, camosic acid, camosine, carotene, carrangeen powder, catalpol, catechins, cellulose gum, ceramide, chitosan, chlorhexidine digluconate, cholesterol, chlorogenic acid, choline, chrysin, chicory, citric acid, coco betaine, cocodimonium hydroxypropyl hydrolyzed rice protein, coco glucoside, coconut endosperm, coenzyme Q10, collagen, corn gluten, extract in CRA, creatine, decyl glucoside, dehydroacetic acid, dipalmitoyl hydroxyproline, dipotassium glycyrrhizate, ECGC (green tea), enzymes, evodiamine, famesol, ferment filtrates, ferulic acid, fibronectin, flavo-glycosides, flavone, flavonoids, folic acid, fungi, ginsenosides, gingerols, glucono-lactone, glucono delta lactone, glucose, glycerin, glycolic acid, glycoproteins, glycyrrhizic acid, glycyrrhizinate, guar gum, honey, honokiol, humectants, hyaluronic acid, hydrolyzed vegetable protein, hydrolyzed sea protein, hydroxycaprylic acid, hyropyltrimonium honey, hypericins, hyperosides, inulin, iridoid glycosides, isoflavones, jojoba protein, keratin, kojic acid, konjac glucomannan, lactobionic acid, lactic acid, lactone, Lactobacillus ferment, Laminaria, Leuconostoc/radish root ferment filtrate, ligustilides, liposomes, lupine protein, luteolin, magnesium ascorbyl phosphate, magnesium chloride, magnolol, malic acid, malts, maltodextrin, mannan, methanol, methyl nicotinate, methylsulfonylmethane, Micrococcus lysate, milks, moisturizing agents, montmorillonite, myristoyl hydrolyzed collagen, morocco lava, myristyl lactate, myristyl myristate, myristyl octanoate, clay N-hydroxysuccinimide, niacine, niacinamide, oat flour, oat protein, octyl dodecanol, oleuropein, oleyl alcohol, olivoyl hydrolyzed wheat protein, oxido reductases, PCA, PCA ethyl cocoyl arginate, palmitoyi oligopeptide, ornithine, palmitoyi tetrapeptide-7, panthenol, pantolactone, papain, pantethine, pectin, pentasodium pentatate, pentylene glycol, peptides, Phaeodactylum tricornutum, phosphatidylcholine, phospholipids, piroctone olamine, bee pollen, polycryalamide, polyvinyl alcohol, pyridoxine HCI, phenslic compounds, Pichia/resveratrol ferment, polyphenol, polyssacharides, Populus tremuloides extract, potassium alum, potassium aspartate, potassium carbonate, potassium cetyl phosphate, potassium chlorate, potassium chloride, potassium cocoate, potassium cocyl hydrolyzed collagen, potassium etecylenoyl hydrolyzed collagen, potassium hydroxide, potassium iodide, potassium olivoil PCA, potassium palmitoyi hydrolyzed wheat protein, potassium persulfate, potassium silicate, potassium sorbate, potassium stearate, potassium sulfide, botanical extract powders, praline, proanthocyanidins, propionic acid, propyl gallate, propylene glycol, propylene glycol alginate, proteins, pseudo-collagen, pullulan, pyredinedicarboxylic acid, pyridoxine, pyridoxine HCL, pyrolidone carboxylic acid, quinine, resveratrol, reticulin, retinol, riboflavin, rice protein, royal jelly, rutin, Saccharomyces cerevisiae, saccharomycesm ferment lysate filtrate, salicin, salicylic acid, salts, schisandrins, Sclerotium gum, sea fennel, sea kelp, sericin, serine, shellac, silica, silk amino acids, silkworm, silt, silver nitrate, silymarin, sine adipe lac, sodium citrate, sodium hyaluronate, sodium hyaluronic acid, sodium bicarbonate, sodium benzoate, sodium bisulfite, sodium carbomer, sodium choride, sodium chondroitin sulfate, sodium citrate, sodium cocoate, sodium dehydroacatate, sodium DNA, sodium formate, sodium gluconate, sodium glutamate, sodium hydrogenated tallow glutamate, sodium hydrosulfite, sodium hydroxide, sodium iodide, sodium isethionate, sodium isostearoyl lactylate, sodium lanolate, sodium lauroyl glutamate, sodium lactate, sodium lauroyl lactylate, sodium mannuronate methylsilanol, sodium metabisulfite, sodium PCA, sodium polyacrylate, sodium propionate, sodium RNA, sodium salicylate, sodium stannate, sodium stearate, sodium stearoyi glutamate, sodium stearoyi lactylate, sodium starch octenyl succinate, sodium sulfate, sodium sulfite, sodium tallowate, sodium thiosulfate, solum diatomeae, solum fullonum, sorbic acid, sorbitol, soy acid, soy protein, Spirulina, steareth-20, succinic acid, sucrose cocoate, sucrose laurate, sucrose polycottonseedate, sugars, sulfur, syrup, tannic acid, tannin, tartaric acid, taurine, tetramine salts, terpene lactones, thiamine nitrate, thiotaurine, Tilia, tranexamic acid, trehalose, triacetin, tricaprylin, tricontanyl PVP, triethyl citrate, triterpene, tetradecyl amino-butyroylvalyl-aminobutyric urea trifluoroacetatetriterpenoids, trihydroxystearin, triisostearin, trisodium phosphate, tyrosine, ubiquinone, undecylenic acid, undecylenoyl glycine, undecylenoyl phenylalanine, urea, ursolic acid, usnic acid, valine, vegetal glycerin, Vitreoscilla perment extract, wheat germ acid, wheat germ glycerides, wheat protein, Wisteria sinesis extract, xanthan gum, xylitol, xylitylglucoside, yashabushi extract, yeast betaglucan, yeast glycoprotein, yogurt, Zanthoxylum alatum extract, zinc acetate, zinc acetylmethionate, zinc chloride, zinc gluconate, zinc glutamate, zinc lactate, zinc PCA, zinc ricinoleate, zinc sulfate, zinc undecylenate and any combination thereof. Further bioactive agent(s) include plant-derived natural products from Aloe vera, Arnica, avocado tree, burdock, leaf of life, katafray, sacred lotus, macadamia tree, field mint, Mimosa tenuiflora, hazel nut, olive tree, blue orchid, Perilla, Quinoa, sandalwood, banana, bocoa, teasel, Centella asiatica, turmeric, harungana, hops, sourwood, maritime pine, white tea, lemon thyme, kangaroo flower extract, mitracarpus, oats, coffee bush, cang zhu, quince, water mint, acerola, Albizia, bamboo, Ginseng, marshmallow, kiwi, moringa, strawberry tree fruit extract, desert date, African dbony, furcellaria, succory dock-cress, sanicle, tamarind and vine flower. Even further bioactive agent(s) include secondary metabolites of plants, including phenylpropanoids and their derivatives, such as simple polyphenolics and aromatic/poly-aromatic polyphenols (flavonoids, stilbenes, curcuminoids, coumarins such as rutin, baikalein, baikalin, verbascoside, cyanidine-3-O-glucoside chloride, psoralen, furanocoumarin, furanocoumarin bergapten, methoxsalen, furocoumarin imperatorin etc.); glycosides (rhamnose, mannose, rutinose, etc.); terpenoids; nitrogen-containing heterocycles, such as alkaloids, purines, pyrimidines, porphyrins, chlorophylls, flavins, etc.

As well as bioactive agent(s) incorporated into solution, the present invention also allows for the presence of further particulates suspended in the liquid mediums.

In a further embodiment, the present invention loads a hydrophobic compound into the polymer mats to increase oil uptake. It has been seen that such a process can double oil sorption. The hydrophobic compound may be added into the oil phase itself such that an increased amount of said phase is absorbed into the mat.

The dry creams and topical delivery systems according to the present invention are made by electrospinning.

In an embodiment, the polymer material is dissolved in a suitable solvent or solvent system in combination with one or more bioactive agent(s) and/or oil(s) to form an electrospinning solution. Further additives as outlined including surfactants and the like may be added to ensure the solution is suitable for electrospinning.

In a preferred embodiment, the formula also included a surfactant or other excipients to achieve a stable process. This may be advantageous with multi-nozzle electrospinning techniques. Polyoxylglycerides (e.g. Labrasol, Labrafil M 1944 CS), polyoxylethylene stearates (e.g. Tefose 63) and poloxamers (e.g. Pluronic F-127) are examples of suitable surfactants but the skilled person will select suitable further excipients as required.

Alternatively, the bioactive agent(s) and/or oil(s) are not incorporated directly into the fibers but are subsequently added. This can be achieved by attaching the bioactive agent to the surface or inside the fibers, for example by spraying.

The fiber mat is collected via a suitable technique, including drum rotating or static collection. In a preferred embodiment, the product is collected on a static collector. In a further embodiment, the product is collected on a plate collector. The plate collector may be static or may have x-y 2D motion or x-y-z 3D motion. Plate collection is preferred over drum collection as the latter resulted in aligned fibers which were mechanically weak.

In an embodiment, the present invention may utilize co-axial electrospinning. For example, with outer PEO and inner SA (although other materials can be used). Tri-axial electrospinning is also encompassed by the present invention. A multi-needle electrospinning head may be used.

During electrospinning, fibers oscillate across the collector, which can result in thicker fibers at the middle of the sheet compared to the edges. Fibers may be combined and mixed to ensure homogeneity.

The mat may be shaped into a suitable form for application by the user.

The mat may be stored in a dried state. This helps to improve shelf life and preserve bioactive agents present. The mat may be wetted to ‘activate’ the topical application properties. Suitable wetting agents may be polar, non-polar or an emulsion. In a preferred embodiment, the wetting agent is water. Alternatively, the wetting agent may itself be a composition that has topical properties, including creams, foams, gels, lotions, ointments and the like.

Thus, in a preferred embodiment, the dry cream or topical delivery system (for example mask) is kept in a dried state until use. When use is required, the mat is soaked in a commercial or premade solution to wet the mat prior to use. An advantage of storing mats in a dried state is an increase in the stability of the bioactive agent(s) and/or shelf life when compared to soaked products currently available.

Alternatively, the dry cream or topical delivery system may be stored in a wetted state ready for direct application.

In an embodiment, the dry cream or topical delivery system has an average thickness of around 500 μm-5 mm, around 1 mm-3 mm, around 500 μm, around 1 mm, around 1.5 mm, around 2 mm, around 2.5 mm, around 3 mm, around 3.5 mm, around 4 mm, around 4.5 mm or around 5 mm. The thickness will be adjusted according to need in terms of mechanical strength and volume of excipient to be carried.

Products of the present invention include products used in cosmetics and personal care. Mats made according to the present invention can be used as masks (e.g. facemasks), dry creams, skin moisturisers, lipsticks, lip balms, lip gloss, nail polishes, beauty serums, lotions, sunscreens, soaps and other body cleansing products, powders and colours for the skin, eyes and lips, skin tanning preparations, oral care preparations, personal hygiene products, perfumes and other aromatic substances. Uses include use as skin moisturisers, anti-aging, anti-redness, anti-acne, whitening. If appropriate, the mat can be shaped for suitability for a particular application. For example, a small patch fiber mat may only cover certain areas of the face or body for, say, anti-acne. The small patch can be made into single dose cream, for example face cream. It can be put on the palm of hand with added few drops of the water or serum and rubbed between hands (e.g. to provide mechanical agitation) to transform the mat to cream/gel consistency and applied on the face as a facial cream. As such, products according to the present invention could be mixed (e.g. mechanically agitated) in situ (e.g. on the face) to change (e.g. disintegrate) into a suitable consistency or could be mixed (e.g. mechanically agitated) in, say, the hand prior to application. Altering the size of the mat and the bioactives/oils it contains and its application method enables a wide variety of potential uses.

Products of the present invention include products used for the topical delivery of bioactive agents for therapeutic use. Bioactive agents include those which have a local effect on the skin and associated tissues, and also systemic bioactive agents. Topical delivery may include application to the skin, vagina, nose, eyes, nails and the like.

In an embodiment, a dry cream or topical delivery system (e.g. mask) is applied to the body (for example skin or face) and is allowed to remain in situ for a period of time to exert a desired effect. This may typically be around 15-30 minutes but longer or shorter times may be envisaged depending on use. The length of time will be selected to enable the one or more active ingredients to impart an effect, for example a cosmetic effect or benefit or a therapeutic effect or benefit.

As explained herein, dry creams and topical delivery systems of the present invention have application as a pharmaceutical delivery system. Topical approaches for formulating drugs rely on the penetration of the drug through layers of skin or mucous membranes; this can decrease systemic side effects.

The present invention can be used as a topical carrier for poorly soluble drugs. The mats can be used for wound treatments or for the treatment of skin infections. Electrospinning as a component of the manufacturing process is quick and cheap which will benefit the manufacturer. Furthermore, the hydrophilic nature of the mats enable easy disposal (e.g. through disintegration), either absorbed into the body or washed away.

The following examples further illustrate the invention. They should not be interpreted as a limitation of the scope of the invention.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

### EXAMPLES

### Materials and Methods

### Materials

Polyvinylpyrrolidone (PVP), Luvitec, grades K60, have been provided by BASF, Ludwigshafen, Germany. Polyethylene oxide (PEO), Polyox, grades 300K and 600K and have been provided by Colorcon, Dartford, UK. Caffeine 98.5% have been provided by ACROS ORGANICS, New Jersey, USA. Olive oil from Fisher Scientific, UK. Ethanol (EtOH), chloroform and Dichloromethane (DCM) have been provided by VWR chemicals, US. Sodium alginate (SA) was a gift from London Metropolitan University or was obtained from Sigma Aldrich.

### Texture Analysis

The mechanical properties of electrospun mats were probed by a texture analyser (TA) Samples were cut in square shape and mounted on top of glass adaptor. Both dry and mats soaked in olive oil were tested. The test was performed with a cylindrical probe (0.6 cm) on TAXT2 instrument (Stable Micro Systems, Surrey, UK). The parameters were set as follow: speed (1 mm/s), a trigger load force (3 g) and a depth (10 mm). Exponent software was used to analyse the data. The tensile property of the electrospun mats was tested on the same apparatus with a cross-head speed of 10 mm/min at constant temperature and humidity.

### Oil and Aqueous Solutions Absorption

The electrospun fibers were cut into pieces of approximately similar size, and precisely weighted. The samples obtained were soaked into two different solutions, olive oil or simulated sweat for 15 min. After that, they were put on top of a sieve until there were no leak and then weighed again.

The “Sorption capacity” was calculated using the Equation (1).

$$Sorption Capacity ( g / g ) = ( Ww - Wd ) / Wd \qquad ( 1 )$$

$$Ww : weight of wet fibers after absorption$$

$$Wd : weight of dried fibers$$

### In Vitro Permeation Study

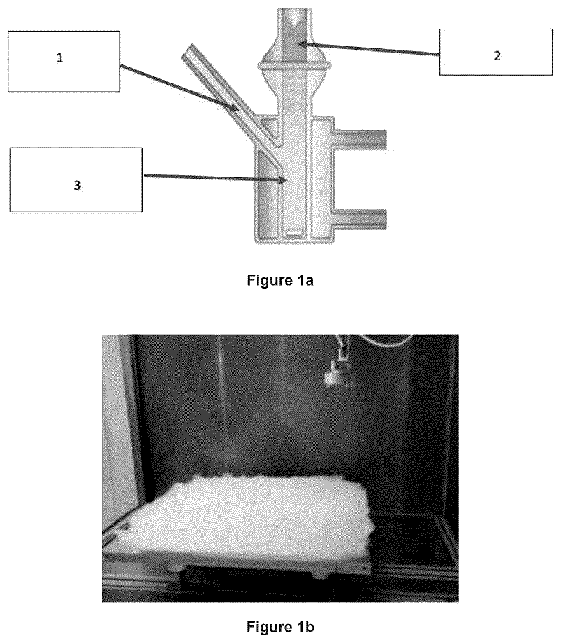

Simulated human sweat (SSW) (pH 5.4) media was prepared using method reported previously (Marques, M. R. C., Loebenberg, R. & Almukainzi, M. Simulated Biological Fluids with Possible Application in Dissolution Testing. Dissolution Technol. 18, 15-28 (2011)). Diffusion experiments were conducted in ‘Franz’ diffusion cells (PermeGear, I. Parts of a Franz Cell. (2017)). The receptor compartment was filled with SSW (pH 5.4) and was continuously stirred (FIG. 1a). (1) shows the sampling compartment, (2) donor compartment, and (3) receptor compartment. Various caffeine solutions with different concentration were applied to the donor compartment.

### Example 1—Preparation of Fibers

F_{PEO} and F_{PS} fibers were prepared by dissolving PEO (6.67% w/v), co-dissolving PEO (5% w/v) and SA (1.5% w/v); respectively in deionised water. F_{PVP} were prepared by dissolving PVP (10% w/v) in ethanol.

The solutions were then loaded into 5 mL syringe and placed on a syringe pump (KDS100, Cole-Parmer, Vernon Hills, IL, USA)., the syringes were fitted with a metal needle tip, and connected to a positive electrode of a high voltage DC power supply (Gamma High Voltage Research, Ormond Beach, FL).

The flow rate was varied between 0.5 to 2 ml/h. Distance was maintained between 10-18 cm and voltage between 12.5 to 23 kV. The grounded electrode was connected to a metal collector (17*17 cm²) or drum rotating at 1000 rpm wrapped with aluminium foil. The humidity of the spinning chamber was maintained around 20% RH during the preparation of F_{PEO} and F_{PS} fibers. The experiments were carried out a between 10-20° C.

An example of fibers made according to the present invention is shown in FIG. 1b.

Exemplary parameters used to make fibers according to the present invention are provided below.

PEO/SA samples were electrospun on a Professional Lab Device form Doxa Microfluidics. To produce the samples, one ten needles multi-nozzle injector was used. Fibers were deposited directly on the surface of a stainless steel flat collector whose size is 500×500 mm. Motion axis were used to obtain a homogeneous sample.
Nozzle to collector distance (cm): 27
Collector type: flat
Flow rate (ml/h): 1.0
HV applied (ΔKV): 31.84
Running Time (hours): 27
Relative Humidity (%): 35-47
Temperature (C): 22-24 (22.9, 23.4 or 24.7).

Further fibers we manufactured together with their characterising data as shown in Table 1 below.

TABLE 1

| | Flow | | | | |
|---|---|---|---|---|---|
| Voltage | rate | Distance | Sorption capacity (g/g) | | Diameter |
| (kV) | (ml/h) | (cm) | Olive Oil | Simulated sweat | (nm) |

PEO fibers (high MW PEO; solvent: deionised water)

| | | | | | | |
|---|---|---|---|---|---|---|
| F_{PEO 1} | 12.5 | 0.5 | 18 | 6.34 ± 0.05 | — | 246.2 ± 81.7 |
| F_{PEO 2} | 17.5 | 0.5 | 18 | 7.55 ± 0.98 | — | 253.8 ± 20.4 |
| F_{PEO 3} | 22.5 | 0.5 | 18 | 9.75 ± 0.44 | — | 235.6 ± 22.9 |

PEO/SA fibers (solvent: deionised water)

| | | | | | | |
|---|---|---|---|---|---|---|
| F_{PS1} | 17 | 0.5 | 13 | 9.48 | — | 239.1 ± 60.3 |
| HMW | | | | | | |
| PEO | | | | | | |
| F_{PS2} | 20 | 0.5 | 13 | 8.98 ± 0.78 | 3.85 ± 0.36 | 232.4 ± 46.6 |
| HMW | | | | | | |
| PEO | | | | | | |
| F_{PS3} | 23 | 0.5 | 13 | 18.75 ± 1.08 | 6.17 ± 0.51 | 240.3 ± 42.1 |
| HMW | | | | | | |
| PEO | | | | | | |
| F_{PS4} | 20 | 0.5 | 10 | 7.97 ± 0.62 | 7.47 ± 0.46 | — |
| HMW | | | | | | |
| PEO | | | | | | |
| F_{PS5} | 20 | 0.5 | 18 | 18.47 ± 1.88 | 11.80 ± 1.0 | — |
| HMW | | | | | | |
| PEO | | | | | | |
| F_{PS6} | 20 | 0.5 | 13 | 21.24 ± 1.14 | 7.44 ± 0.61 | 172.6 ± 36.9 |
| LMW | | | | | | |
| PEO | | | | | | |

PVP fibers (solvent: ethanol)

| | | | | | | |
|---|---|---|---|---|---|---|
| F_{PVP 1} | 17.5 | 2 | 18 | 15.34 ± 1.91 | — | 369.2 ± 98.1 |
| F_{PVP 2} | 17.5 | 1 | 18 | 18.92 ± 0.53 | — | 289.1 ± 75.8 |
| F_{PVP 3} | 15 | 1 | 15 | 19.76 ± 0.05 | — | 264.0 ± 81.7 |

### Example 2—Morphological Properties

The morphological properties of samples made according to Example 1 were characterized using scanning electron microscope (SEM: JEOL, Japan). Samples were sputtered with a gold coating (5 nm thickness) prior to SEM analysis. The images obtained were then analysed using the ImageJ software and the fiber diameters (based on >50 measurements) were calculated.

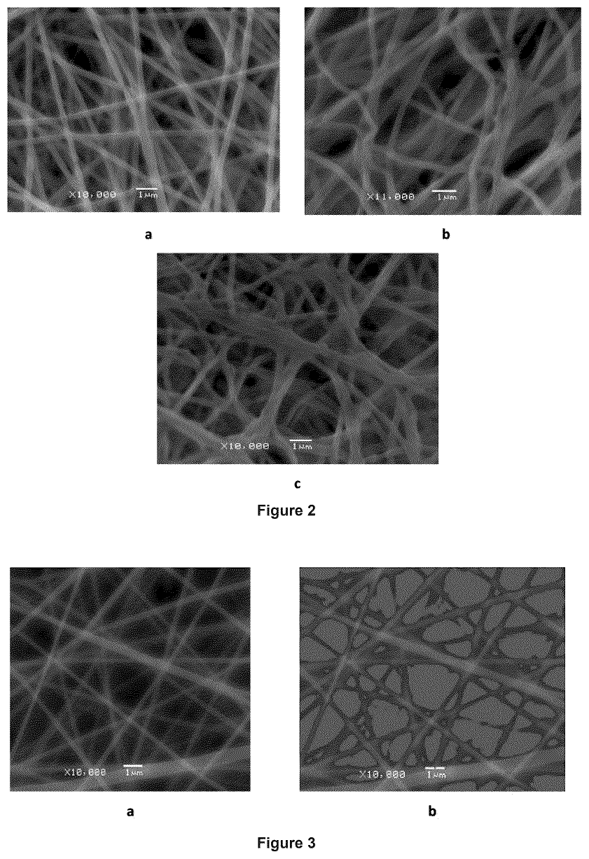

SEM images of three F_{PEO} samples are shown in FIG. 2. It can be seen that the electrospun fibers are smooth and cylindrical. Smooth fibers help facilitate a good skin feel during use making the resultant mat a positive user experience. These fibres are also highly porous and enable resultant mats to have a high absorbing and holding capacity. This allows thin mats to soak up a considerable amount of liquid medium.

### PEO Fibers

It can be seen from Table 1 and FIG. 2 that varying the voltage did not affect F_{PEO} fiber diameters. In all cases, diameters were around 250 nm. However, it was observed that under increasing voltage the pore distribution becomes less uniform and beaded fibers start to occur. See, for example, FIGS. 2b and 2c vs 2a. This may explain the increased oil update correlation to applied voltage. However, less uniform pore distribution and/or beaded fibres may reduce the mechanical strength of the mats.

### PEO/SA Fibers

Adding to SA to PEO did not have any noticeable effect on the fibers diameters, which were similar to PEO fibers. Furthermore, varying the voltage had almost no impact on fiber diameter. However, using low molecular weight (PEO Mw: 300.000) had a significant impact on fibers diameters, it decreased from 232 nm to 172 nm. This may be due to reduced viscosity of the LMW PEO.

### PVP Fibers

For the PVP fibers, different distances, flow rates and voltages were evaluated. It can be seen that decreasing the flow rate improved the absorption capacity. On the other hand, changes in voltage or distance did not significantly affect the absorption capacity.

### Example 3—Oil and Aqueous Solutions Absorption

The oil uptake for materials made according to the present invention is shown in Table 1. It can be seen that all samples demonstrated oil uptake. Uptake varied from 3.37 to 19.76 g/g of their dried weight so it can be seen that all samples were able to uptake several times their weight in an oil. This property is advantageous since it allows hydrophobic solvents to be taken into hydrophilic fibers. This effect is believed to be due to absorption, adsorption, and/or capillary action. Thus, the present invention allows for the uptake of organic material into hydrophilic fibers.

As can be seen from Table 1, the PEO, PEO/SA and PVP fibers showed different oil uptake. PVP achieved the highest uptake, followed by PEO then PEO/SA. This may be due to the pore structure or surface chemistry or charge of the PVP fibres.

A correlation between oil uptake and fiber diameter is also revealed. It can be seen with PVP fibers that a reduction in fiber diameter led to an increase in oil uptake. This may be due to the pore structure (size and/or connectivities between pores).

It can also be seen that with PEO fibers, although the diameters were almost constant, there was nevertheless as difference in oil update. It is believed this could be due to intra-fiber voids that are influenced by the applied voltage. See FIG. 2. As such, voltage can be adjusted to achieve the desired oil uptake properties.

PEO/SA showed higher oil uptake properties to PEO alone. PEO/SA also showed good aqueous absorption.

Aqueous absorption was also assessed using simulated sweat. The aqueous uptake from the various FPS mats was between 4 to 11 g/g. As such, the mats according to the present invention achieve meaningful uptake of hydrophilic material. This means that the present invention can be used to deliver hydrophilic compounds.

The data in Table 1 illustrate F_{PS6} (with lower PEO molecular weight) had a slightly higher uptake than F_{PS2} (higher PEO molecular weight). Uptake properties can be modified by selecting polymers with a particular molecular weight.

Experiments (data not shown) were conducted with identical conditions but using different solvents, chloroform and DCM. No impact in oil updated was identified.

Mats according to the invention are shown to have good uptake of both aqueous and non-aqueous solvents. This means that the mats can be utilised with a range of materials that are hydrophobic, hydrophilic or contain a mix of hydrophobic and hydrophilic materials (for example creams). This provides significant versatility with the materials according to the present invention.

### Surface Area and Pore Size

Surface area and pore size was considered in further detail.

BET surface area analysis technique was employed to measure the surface area and pore size of an F_{PS2} mat. Prior to the measurement, fibres sample (60 mg) was put into a sample holder, then degassed at 25° C. and 10″5 Torr pressure for at least 48 h. The Brunauer-Emmett-Teller (BET) technique was used to characterise the surface area, pore volume, and diameter of the fibres by N2 adsorption at 77.350K with the Quantachrome NovaWin Instrument. The pore size distribution and pore volume were determined by using Saito-Foley (SF) model.

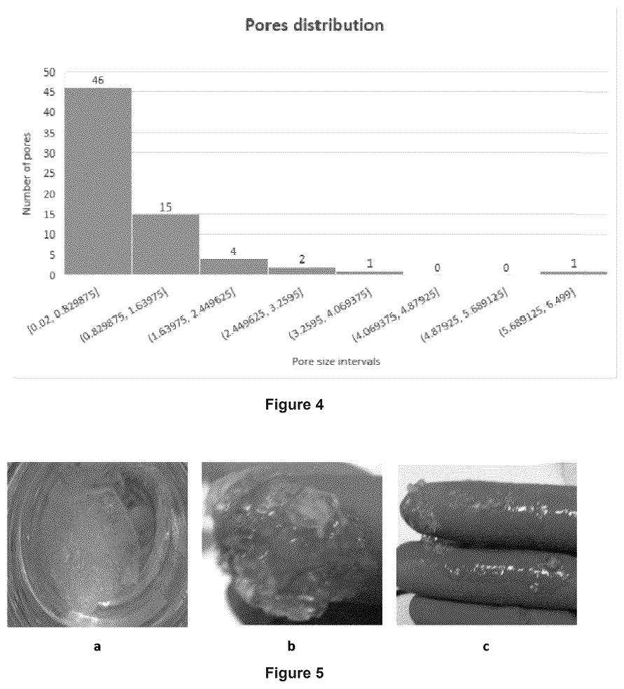

The total pore volume (TPV) was around 0.579 cc/g and the average pore size around was around 0.612 μm. The same sample was characterised by SEM and pores size were measured using ImageJ software and data showed that the mean pore size was 0.780±1.03 μm (FIGS. 3 and 4), which is similar to the BET measurement.

The olive oil uptake by F_{PS2} was measured from the samples that were investigated by the BET. Based on theoretical calculation the olive oil (0.918 g/ml) uptake should be 0.0172 g (0.0187 ml), however, the experimental measurement showed that it was 5.5 times higher than theoretical value (Table 2). As such, the fibers made according to the present invention are able to hold higher volume of liquids than is expected.

TABLE 2
Summary of FPS theoretical and experimental data

| Dry | Wet | Weight | Weight of olive | Volume of olive |
|---|---|---|---|---|
| fibers | fibers | difference | oil absorbed (g) | oil absorbed (ml) |
| (g) | (g) | (g) | Obsd (calcd) | Obsd (calcd) |
| 0.0324 | 0.1287 | 0.0963 | 0.0963 (0.0172) | 0.105 (0.0187) |

It is believed that a swelling process of the F_{PS2} mat took place during oil uptake. The mechanisms of oil uptake by the F_{PS2} mat is believed to be a combination of absorption, adsorption, and capillary action.

### Example 5—Mechanical Removal of Mats

The mechanical agitation and removal of mats was demonstrated. The polymers of the present invention are hydrophilic and soluble in aqueous solutions. A PEO/SA mat (F_{PS2}) was tested for its aqueous solubility and properties under agitation. The results are shown in FIG. 5, which show photographs of the F_{PS2} mat (a) inside simulated sweat, (b) after removal from media; (c) after rubbing.

When placed in an aqueous solution, the mat formed a gel film due to ionotropic gelation that took place between divalent metals and SA. It can be seen from FIG. 5a that the formed gel firm did not dissolve.

Upon removal from the aqueous solution, the mat retained its shape as is shown in FIG. 5b. The effects of rubbing the film is shown in FIG. 5c, and it can be seen that the mat breaks down. This property allows for easier removal post use.

### Example 6—Mechanical Properties of Electrospun Mats

It is crucial that the mats of the present invention retain sufficient strength and shape stability during manipulation. The mechanical properties of masks made according to the present invention were investigated.

All the samples demonstrated a non-linear elastic behaviour during the test.

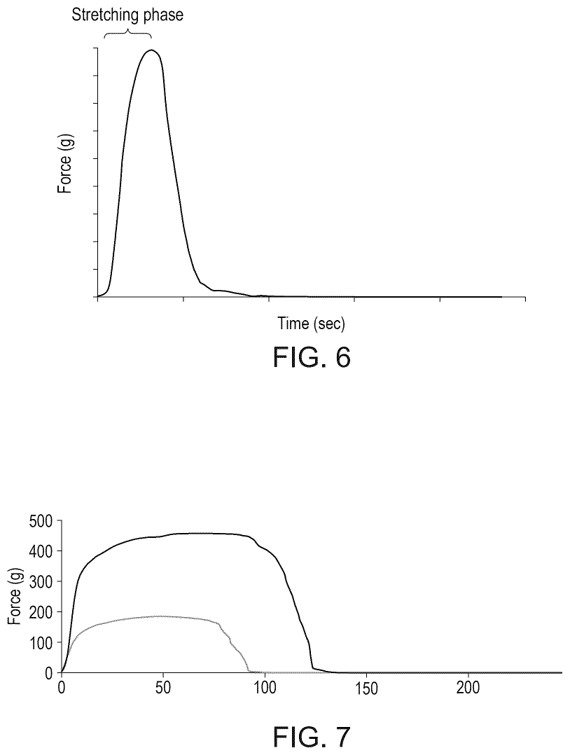

FIG. 6 shows a stress-strain curve of the F_{PS} mat. The mat showed an increased tensile stress till it reached a stabilized higher stage or point. This was then followed by a decrease with no stable phase seen. FIG. 7 shows stress-strain curve of the F_{PEO3} (top line) and F_{PEO2} (bottom line) mats. Again, the mat showed an increased tensile stress till they reached a stabilized higher stage or point followed by a plateau and then a decrease.

The F_{PEO} materials were shown to have elastic properties which was reflected in the first stage of the test.

Then F_{PEO} lost their elastic property but kept stretching and this resulted in the plateau. The decreased in the stress resulted from the fibers breaking down.

The tensile strength increased in the following order: PEO>PEO/SA and these results can be seen in Table 3.

TABLE 3
Summary of mechanical properties of various electropsun mats.

| | Punch | stretch |
|---|---|---|
| Samples | Force (g) | Force (g) |
| Parafilm | 608.9 | 1170 |
| Al foil | 891 | 4400 |
| Roll paper | 59.2 | 1100 |
| F_{PEO 1} | 1024 ± 247 | 160 ± 52.3 |
| F_{PEO2} | 1378 ± 141 | 280 ± 56.5 |
| F_{PEO3} | 1681 ± 485 | 450 ± 83.3 |
| F_{PS3} | 280 ± 75 | — |
| F_{PS5} | 600 ± 282.8 | 300 ± 104 |
| F_{PS6} | 710 ± 276 | 900 ± 275 |
| F_{PS2} soaked in olive oil | 194.9 ± 45 | — |
| F_{PS4} soaked in olive oil | 662 ± 300.5 | — |
| F_{PS5} soaked in olive oil | 250 ± 42 | — |
| F_{PS6} soaked in olive oil | 570 ± 212 | — |

It can be seen that the strength of PEO fibers mats showed positive correlation with the applied voltage. Since PEO fibers generated with various voltage showed almost same diameters, it is believed that higher voltage may lead polymer chains to get more elongate (disentangle).

### Example 7—In Vitro Permeation Study

Caffeine is found in a number of commercial cosmetic formulations and therefore it was selected and used as a model water-soluble compound to examine the effect of fiber formulation on permeation performance. Silicone membranes were used to represent the barrier function of skin. This is a widely used artificial skin model for permeation assessment in transdermal formulations. Caffeine is a rather hydrophilic permeant and it is expected to permeate via polar channels at the surface of the skin. Since caffeine has relatively high water solubility, a reasonable flux through skin is expected.

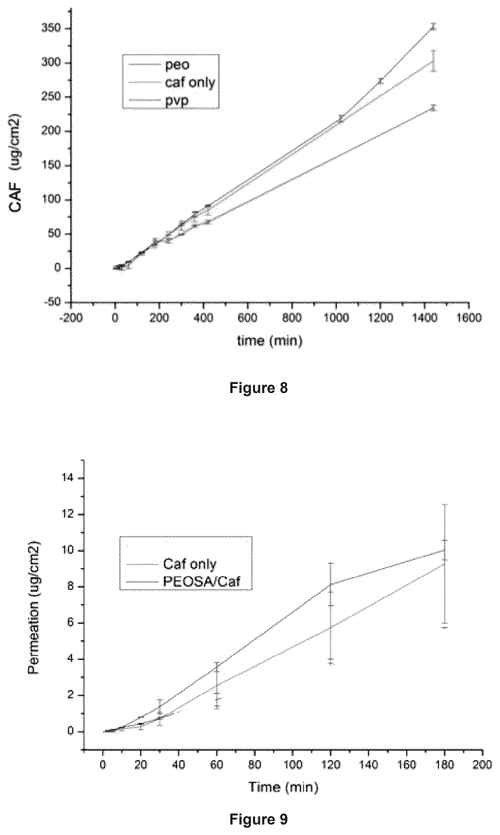

The in vitro study shows that the flux of a caffeine is related to its applied concentration. During the first 3 h, the caffeine permeation from PEO and PVP soaked into caffeine solution and caffeine solution showed no difference (FIG. 8). The difference between the 3 formulations start after 4 h, where PEO fibers showed they improve permeability of caffeine compared to caffeine solution on its own, and PVP slightly reduces caffeine permeation vs control but still results in high caffeine permeation. The effect of PVP on permeation could be due to hydrogen bonding between PVP and caffeine, which can slow the permeation into artificial skin membrane. PEO demonstrated better permeation profile after 4 h than caffeine solution and PVP. As such, it is possible to select a suitable polymer depending on the desired permeation properties.

In a further in vitro study, the permeation properties of caffeine soaked PEO/SA were assessed versus a control sample (CAF solution 0.1 ml at 4.45 mg/ml). The results are shown in FIG. 9. The PEO/SA mat had improved permeation versus the control caffeine solution quickly and certainly after 10 minutes. It can be seen that permeation improvements can be seen with mats according to the present invention over short timescales, in particular timescales that would be used during cosmetic application.

Given the mats of the present invention can uptake both hydrophilic and hydrophobic compounds, hydrophobic permeation is also possible with mats according to the present invention.

PEO masks showed the most advantageous mechanical properties and permeation profile of the polymers tested.

### Example 9—Therapeutic Bioactives

### Preparation of the Polymer Mat

A stock solution of ethanol and dichloromethane (DCM) with the ratio of 7:3 v/v was prepared and used as the solvent in a 10% stock solution of HPMCAS-PVP (at weight proportion of 1:1, 5% each), which ensures quick solvent evaporation. The polymers were precisely weighed out and dissolved in ethanol-DCM. This solution was stirred and sonicated until completely dissolved. This was repeated to prepare a 10% stock solution of PVP. A Cole-Parmer Syringe Pump, UK was used to infuse the polymer stock solutions at a feeding rate of 1.5 ml/hour to a custom-made single spinneret electrospinning collector (covered with aluminum foil) under high voltage (Gamma High Voltage Research, Ormond Beach, FL) of 15 kV. The drum rotation rate of the collector was 500 rpm and placed at a distance of 15 cm from the infuser. A 5% stock solution of PEO was made up with distilled water. This was electrospun at the same conditions as HPCAS-PVP and PVP except that the solution feeding rate was 0.5 ml/hour at 16.5 kV.

### Drug Loading

Metoprolol has a solubility of 31 mg/ml in ethanol. The maximum amount of metoprolol which can dissolve in the volume of ethanol in a 10% solution was precisely weighed out and dissolved in the ethanol—DCM in the HPMCAS-PVP polymer solution before it was electrospun. When coumarin (hydrophobic fluorescent dye) was added, the voltage was increased to 19.1 kv. Coumarin and metoprolol together increased the surface tension of the solution so a higher voltage was required to overcome this. Ketoconazole was dissolved in sunflower oil. The solubility of ketoconazole in sunflower oil was approximately 5.8 mg/ml.

### Characterization of Structured Oil Systems Oil Absorption Test

The resulting dry electrospun fiber mats were then cut to size, precisely weighed and soaked in a quantity of oil which was calculated to be 100 times heavier than the fiber mats dry weight. The oil soaked fiber mats were re-weighed after 2 hours to distinguish how much oil was up taken by the fiber mats. A calculation was performed to distinguish how much oil the polymer mats had taken up in comparison to its original dry weight. The hydrophilic drug (metoprolol succinate) loaded polymer fiber mats were soaked in placebo sunflower oil. Placebo HPMCAS-PVP bend fiber mats were tested with the hydrophobic drug (ketoconazole) loaded sunflower oil.

### Texture Analysis

A TA-XT2 texture analyzer from Stable Microsystems (Godalming, Surrey, UK) was used to measure the mechanical properties of the fibers. Texture analysis relies on the homogeneity of the fibers which is why the center of the fiber sheets produced was used. The edges of the fiber sheets are thinner than the center. The fibers were cut to a similar size and clamped down using a 9 mm glass adaptor. A % inch spherical stainless probe was used to penetrate the polymer fiber mats until the polymer network had broken. The amount of force required to break the polymer mat was recorded. Both dry and fibers soaked in oil were tested. To test self-healing properties, a stainless steel 1 mm needle (point tapered 2-3 mm) was used and passed though the polymer mat three times, through the same hole with and without oil. Speed of probe at all times was 1 mm/second. The adhesion of the polymer mats was measured by lowering the % inch spherical stainless probe down so that it only touched the surface of the fiber mats. The stickiness of the polymer mats to the probe was measured.

### Microstructure Studies

Coumarin was dissolved in the polymer solution and then was electrospun to give fluorescent fibers. A Zeiss Axiovert 200M widefield 2 fluorescent microscope (Carl Zeiss Ltd., Cambridge, UK) was used to view the fibers with and without oil. A Zeiss M2 Bio Quad SV 11 stereo microscope (Carl Zeiss Ltd., Cambridge, UK) was used to view self-healing properties. The fiber mats were pierced with a stainless steel 1 mm needle (point tapered 2-3 mm) and then the fiber structure were viewed. A scanning electron microscope JEOL JSM 5900LV (Tokyo, Japan) was used to view the morphology of the dry fiber mats and to measure the fiber diameters. A small section of the polymer mat was placed onto the sample stab and coated with Au/Pd using a Polaron SC7640 Quorom Technologies gold sputter coater, before viewing the image. More than 90 fiber diameters measurements were measured from three different SEM images for each polymer using Image-J software. The average diameter and standard deviation were calculated for each polymer. The A LEICA MZ7.5 stereo microscope was used to view the surface of the fibers with and without oil and when it was pierced.

### Differential Scanning Colorimetry (DSC)

A Q-2000 (TA instruments, Newcastle, DE, USA) was used to perform DSC experiments. n-Octadecane, indium, and tin were used for temperature calibration. The heat capacity in modulated mode was calibrated using a sapphire calibration disc (TA instruments, Newcastle, DE, USA). The heating rate was at 2 C/min with modulated amplitude of +/−1° C. every 60 see from 0 to 200° C. The samples were precisely weighed between 2-4 mg in standard TA aluminum crimped pans.

### The Effect of Water on Polymer Network

A polarized light microscope model Leica DM LS2 (Wetzlar GmbH, Germany) fitted with a JVC camera with 5, 10 and 20 magnification was used to view the impact water had on the polymer fiber mats once soaked in oil. An oil-soaked fiber mat was placed on a glass slide. A droplet of water was placed on one corner of the mat, allowing it to migrate across the polymer mat. This was viewed and recorded on the microscope. This is important because the polymer blends contain hydrophilic polymers whose physical properties may change if exposed to moisture.

### Oil-Absorbing Capacity of Placebo Fiber Mats.

Oil sorption tests were conducted to determine the oil sorption capacity of HPMCAS-PVP, PVP and PEO polymer mats. The average uptake of oil by the HPMCAS-PVP fiber mats was lower than the PVP fibers as shown in Table 4.

PEO was tested with 40 times its dry mass of oil because its oil sorption was found to be approximately 40 fold its dry mass during initial observations. The PEO values suggest that they were similar to the blend fibers, as the average sorption was 36.2 times its dry mass (Table 1). The percentage of sorption ranged between 83-99% of the total quantity of oil added.

Coumarin was incorporated into the fibers as a fluorescent dye which is used in identifying fiber morphology. The fluorescent fibers were tested for their oil sorption ability to observe whether coumarin affected the sorption of the fibers. HPMCAS-PVP fiber mat prepared with coumarin sorbed an average of 67.3 times its mass in oil which was greater than the PVP fibers; 58.2 times its weight in oil (Table 1). It was hypothesized that coumarin contains hydrophobic groups which would have a higher affinity to the oil leading to higher oil uptake into the fibers.

TABLE 4
oil sorption results of placebo polymer fiber
mats and the drug loaded polymer fiber mats

| | Average oil- |
|---|---|
| | sorption (fold of |
| Sample: | oil:dry mass) |
| HPMCAS-PVP | 37.125 ± 1.658 |
| PVP | 59.25 ± 12.897 |
| PEO | 36.24 ± 2.691 |
| HPMCAS-PVP with Ketoconazole | 78.9 ± 2.981 |
| HPMCAS-PVP with Metoprolol | 39.53 ± 3.453 |
| HPMCAS-PVP with Coumarin | 67.33 ± 4.565 |
| PVP with Coumarin | 58.23 ± 11.651 |

### Effect of Drug Incorporation on the Oil-Sorption Capacity of Fiber Mats

Due to the polymer mats potential application as a topical formulation, a hydrophilic (metoprolol) and hydrophobic drug (ketoconazole) was loaded into the fiber mats to determine their effects on oil sorption.

The nature of the fiber mats was influenced by the incorporation of a drug into the polymer network. Metoprolol succinate was added into the polymer solution before electrospinning due to its higher solubility in ethanol. The sorption of the metoprolol loaded fiber mats was comparable to the volume of oil up taken by placebo HPMCAS-PVP fibers (Table 1). Metoprolol can form hydrogen bonding with PVP in the blend via C═O, N—H and O—H groups. However, this does not influence the volume of oil being drawn into the fibers. There are not a significant number of hydrophobic groups in metoprolol to increase its affinity to oil. Therefore, this may explain the similar values.

Ketoconazole was dissolved in sunflower oil due to its hydrophobicity. This solution was used to test ketoconazole's effect on placebo HPMCAS-PVP oil sorption. The average oil sorption of the ketoconazole loaded HPMCAS-PVP fibers was almost double the oil sorption capacity of metoprolol loaded fiber mats. Without wishing to be bound by theory, the hydrophobic groups in ketoconazole may have a high affinity to the hydrophobic entities in HPMCAS such as C—H and CH2, which draws the higher volume of oil into the polymer network via capillary action.

### Physical Properties of the Fiber Mats

To estimate the miscibility between the components in the fibers as a bulk mat, DSC was performed on the individual polymer mats as well as the placebo and drug loaded polymer blend mats.

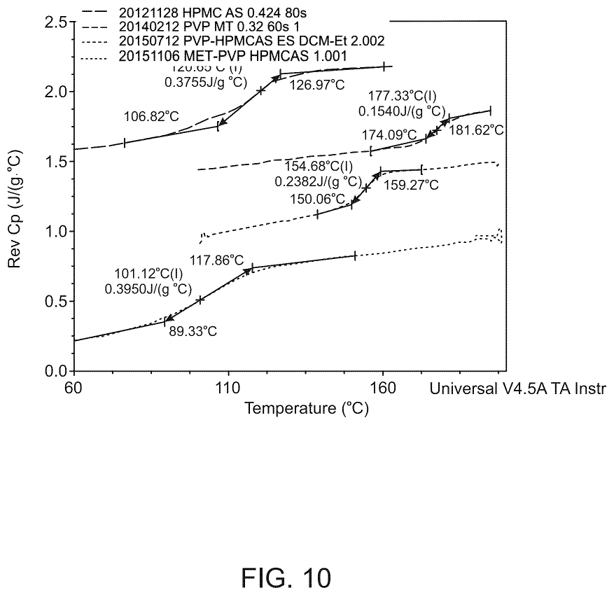

The DSC results for the individual polymer fiber mats in FIG. 10 shows a single Tg, of 120.65+0.37° C. and 177.33+0.15° C. for HPMCAS and PVP respectively, which is expected for amorphous materials. HPMCAS-PVP and HPMCAS-PVP with metoprolol fiber mats also have a single Tg at 154.68+0.24° C. and 101.12+0.39° C. respectively, but also have a melting point peak.

The placebo HPMCAS-PVP fiber mats had a Tg which was intermediate to the Tg values of the individual polymers suggesting full miscibility of the polymer blend (26).

The lowered Tg for the drug loaded polymer mat demonstrates a plasticizer effect due to the presence of metoprolol in the blend polymer mats. The melting enthalpy for the drug loaded fiber mat was approximately 200° C., suggesting that the crystalline metoprolol is melting. This indicates low miscibility between the components which make up the fiber mats.

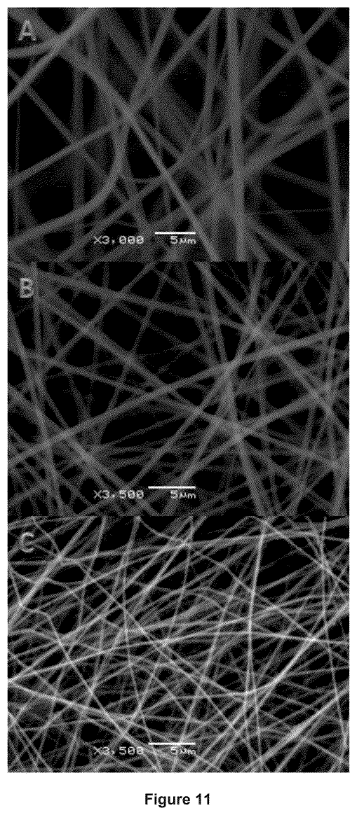

SEM was used to determine the morphology of individual fibers and to observe structural features which could explain the mechanism of oil sorption. The morphology of the electro-spun fiber mats can be seen to have smooth surfaces with no beading (FIG. 11).

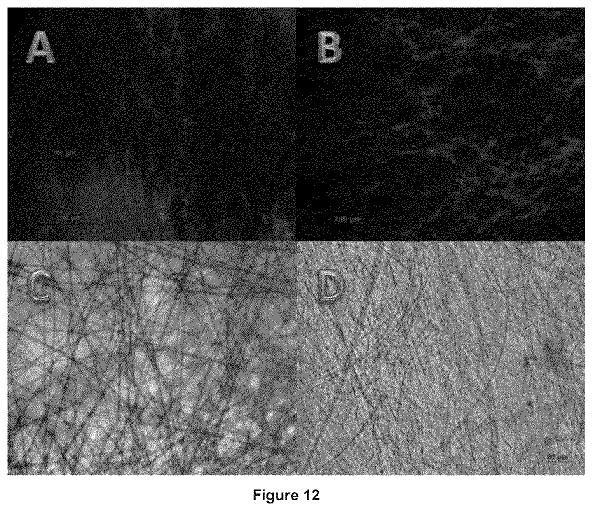

Diameters were obtained from analysis of SEM images. The average sizes of HPMCAS-PVP fibers (1.05+0.3 μm) were greater than PVP (0.60+0.13 μm) and PEO (0.24+0.05 μm). The molecular weight of the polymer, its surface tension and evaporation rate may have influenced polymer diameter. From the fluorescent images it appears that the oil-soaked fiber mats are the same size and thickness as the polymer mats without oil (FIG. 12). This implies that the fibers do not swell with oil. The oil appears to be physically trapped within inter-fiber spaces in the polymer network. The oil may be adhering to the surface of the fibers; though there are no obvious interfaces between the oil and the fiber. The fluorescent images showed similar results across all fibers mats including the drug loaded mats.

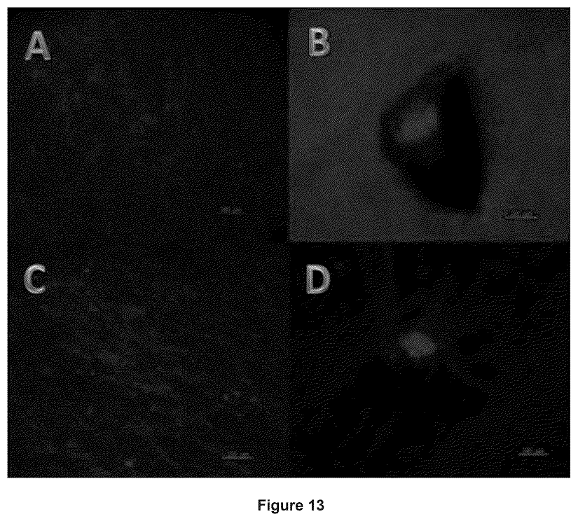

Macroscopically, the oil-soaked fibers appeared to self-heal when they were pierced with a needle. This assumption was tested by observing the polymer network under a fluorescent microscope once the mat was pierced. The images obtained show the individual fibers and how they assemble before and after being pierced (FIG. 13). It appeared that the HPMCAS-PVP blend fibers coiled upwards once the needle had exited the fiber (FIG. 13D).

Texture analysis was used to measure the force required to pass a needle probe through the pierced fibers. If further force was required to repeatedly pierce the mats then the fibers could possibly have self-healed. Since there was no force required to pierce both the HPMCAS-PVP and PVP it could therefore be suggested that the broken polymer network would not repair itself. The macroscopic observation of self-healing is due oil filling the broken network (FIG. 13D).

### Mechanical Properties of Fiber Mats

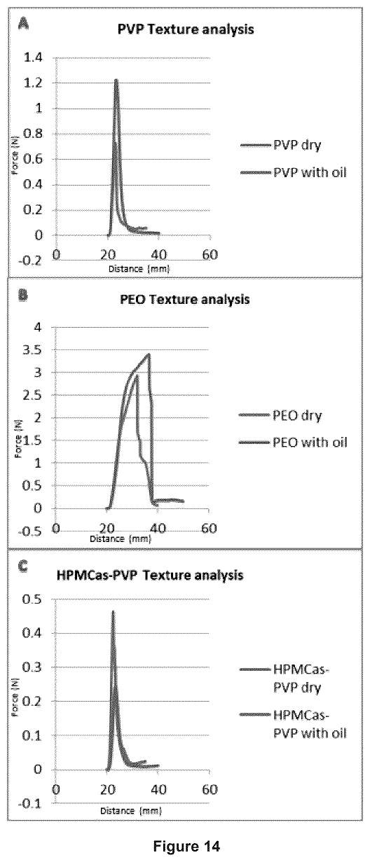

The initial force (N) required to break the dry fibers is the physical representation of the strength of the film. The force required to break the dry fiber mat was highest in the HPMCAS-PVP blend at approximately 4.6N compared to PVP (0.7N) and PEO (2.9N) (FIG. 14). Approximately half the force (2.4N) was required to break the HPMCAS-PVP fibers after being soaked in oil. Conversely the values for PVP (1.21N) and PEO (3.4N) are higher when soaked in oil (FIG. 14A, 14B). The difference in these values could be attributed to the varying diameter of the different fibers. PEO having thinner fibers may make the fiber mat less rigid. This would increase the force required to break it. The lack of a definitive peak upon breaking suggests the fiber mat is elastic (FIG. 14B).

HPMCAS-PVP fiber mats become weaker when soaked in oil. A possible reason for this is that the triglycerides present in sunflower oil may be disrupting the interaction between HPMCAS and PVP. This is possibly because the hydrophobic-hydrophobic attraction between HPMCAS and oil was stronger than the interaction between the blend polymers which may be the reason the polymer network had weakened in the presence of oil. More force is required to break oil-soaked PVP and PEO fiber mats because the elasticity and flexibility of the fiber mats may have increased by the oil settling in between the individual fibers.

Variance of the electrospun fibers made texture analysis comparisons difficult. Steps were taken to increase homogeneity. During electrospinning, the fibers oscillate across the collector, resulting in thicker fibers at the middle of the sheet compared to the edges. However, the fiber mats are not uniform across the sheet. The centre of the fiber mat sheets were used to increase homogeneity. Physically measuring the thickness of the fiber mats was difficult; therefore, each cut section was individually weighed to compare homogeneity. Each polymer was tested 5 times and anomalies were discarded during the calculations in order to increase replicability.

The area under the curves equates to the work (J) required to pass the probe through each fiber. It also contributes to the work required to stretch the fiber, deform and to break it. The amount of work required to break each polymer mat with and without oil corroborates with the data showing the force required to break the fiber mats.

The work required to break the PEO fiber mats was significantly higher than PVP and HPMCAS-PVP fiber mats. This could be due to the thickness of the bulk mat and the diameter of the individual fibers. The trend suggests that the smaller the diameter, the stronger the fiber mat, although this does not take thickness of the mats into account.

There were some differences in the shape of the graphs produced due to the fiber not breaking cleanly or if the fiber was not uniform. HPMCAS-PVP and PVP fiber mats broke more cleanly suggesting a more rigid polymer network even with oil. PEO however had a less defined peak when breaking implying the polymer mat stretched before it finally broke.

Adhesion of HPMCAS-PVP and PVP fibers to a metal probe, with and without oil, was tested and compared. The steel probe broke the fiber mats and adhesion to the fibers was measured as the probe was removed. It was apparent that there was some adhesion to all three fibers when they were dry, potentially due to electrostatic attractions of the broken electro-spun fibers to the metal probe.

Conversely, there was no adhesion in all cases between the oil-soaked fibers to the metal probe. The fact that the polymer network was rigid and that the oil was entrapped in the voids of the polymer fibers led to the assumption that the metal probe was being lubricated by the oil and therefore no adhesion was recorded.

The absence of adhesion was also recorded when the metal probe only touched the surface of the polymer mat, with and without the presence of the oil. The difference between the two methods' was that there was adhesion when the polymer network was broken however this was not the case when the polymer remained intact.

Overall, Example 9 shows electrospun polymer networks as a unique innovative technique to structure oil as a therapeutic delivery vehicle. All three placebo polymer mats absorbed similar volumes of oil. Loading a hydrophilic drug into the polymer network did not have a significant effect on its sunflower oil uptake. However, loading the oil with a hydrophobic drug almost doubled the oil uptake. The fiber mats do not self-heal once the polymer network has been damaged (pierced) but can be washed off or absorbed with water.

### Example 10—Bilayer Mask

Sodium alginate (0.5% w/v) and PEO (5% w/v) with molecular weight of 600 KDa were mixed in MQ-water to form the electrospinning solution for an inner mask layer. Zein (25% w/v) and PEO (0.5% w/v) with molecular weight 900 KDa were dissolved in ethanol/water mixture to form the electrospinning solution for the backing layer. The inner alginate layer was electrospun first, followed by the electrospinning of the backing layer. An electrospinning voltage of 18 Kv and 17 Kv were used for the inner and backing layer, respectively. For both layers a flow rate of 0.5 mL/h, a 22 G nozzle and a 20 cm nozzle to collector distance were used.

The performance in terms of burst strength, thickness, oil sorption capacity and water sorption capacity of the bilayer mask was assessed and these are shown below (Table 5). Data for the inner mask layer and the backing layer are also shown.

TABLE 5
Performance of bilayer mask

| | | | Oil sorption | Water sorption |
|---|---|---|---|---|
| | Burst | | capacity | capacity |
| Film | strength (g) | Thickness (μm) | (mg/mg) | (mg/mg) |
| Tissue | 184.44 ± 11.07 | 116.00 ± 20.63 | — | — |
| SA/PEO (0.3 mL) | 159.90 ± 20.91 | 16.33 ± 3.21 | 5.85 ± 2.09 | (not determined) |
| Zein/PEO (1.5 mL) | 343.92 ± 22.84 | 125.67 ± 26.27 | 2.05 ± 0.46 | 1.27 ± 0.19 |
| Bilayer | 217.28 ± 25.11 | 101.00 ± 6.08 | 2.41 ± 0.38 | 1.08 ± 0.34 |

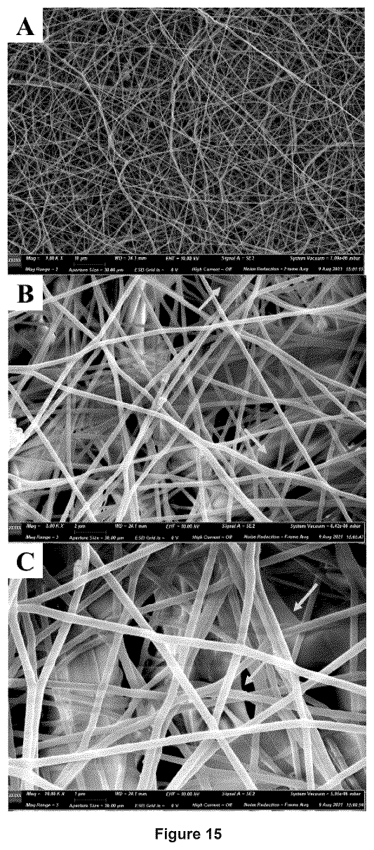

The bilayer mask was also investigated for its response to wetting using microscopy. Prior to wetting, the bilayer mask had intact fibres in both the inner mask layer (SA/PEO) and the backing layer (zein/PEO) (FIG. 15).

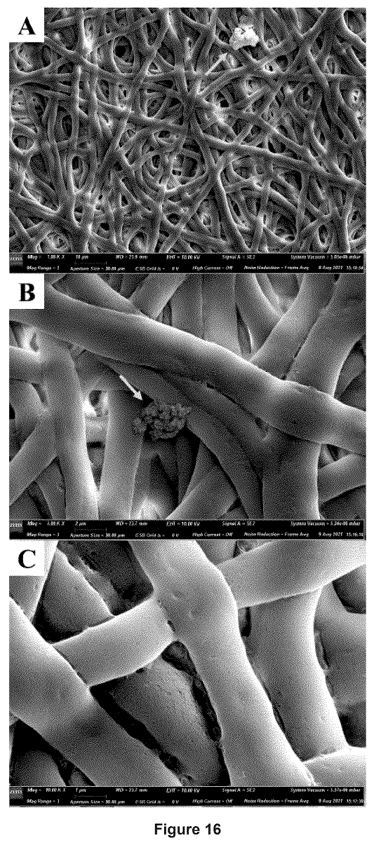

The bilayer mask was then subjected to wetting with water, and then was dried at room temperature. Microscopy revealed that the inner mask layer (SA/PEO) formed particles, but did not dissolve (FIG. 16). The backing layer (zein/PEO) retained a fiber structure even after wetting.

### Example 11—Mats for Dry Cream

An oil-in-water (O/W) emulsion was prepared as the base of electrospinning solution. A wide range of synthetic and natural oils can be used as the oil phase, such as, but not limited to, castor oil and peppermint oil. MQ water containing polymeric stabilisers were used as the aqueous phase. Emulsifiers such as but not limited to Tefose 63 and Labrafil M 1944CS were added into the mixture to form an emulsion. Polymeric stabilisers such as but not limited to Xanthan gum, gelatin, pectin, polyvinyl alcohol, polyvinylpyrrolidone, hydroxypropyl methylcellulose, hydroxypropyl cellulose and polyethylene oxide were suitably added into the emulsion to prepare a homogenous spinnable solution under magnetic stirring condition. The emulsion containing the polymeric stabiliser was subjected to electrospinning using an electrospinning setup at a vertical configuration. A syringe pump was utilised to ensure a constant flow rate of prepared solution. Flow rate was adjusted accordingly to form a stable Taylor cone at an applied electrical potential. For example, for the PVA-HPMC example, a 20 G nozzle with a voltage of 25 kV and 1 mL/h flow rate were used for the electrospinning process. The plate collector covered with aluminium foil was used for the fiber collection. Example formulations of mats for dry creams are described below.

### Example 11-1: PVA/PVP

Conditions: Nozzle—27 G; Flow rate—0.6 to 1.0 mL/h, Voltage—25 to 27.5 kV; Distance—20 cm; drum collector

### Composition of Oil-In-Water Emulsion:

TABLE 6
Composition of PVA/PVP-based dry cream

| Ingredients | Amount |
|---|---|
| Castor oil | 0.1 g |
| Tefose 63 (Gattefossé) | 0.6 g |
| Labrafil M 1944 CS (Gattefossé) | 0.3 g |
| PVA (MW 85000-120000, 88% hydrolyzed, ACROS) | 0.5 g |
| PVP K30 (MW 44000-54000 g/mol, BASF) | 0.5 g |
| MQ water | 10.0 g |

### Fibre Morphology:

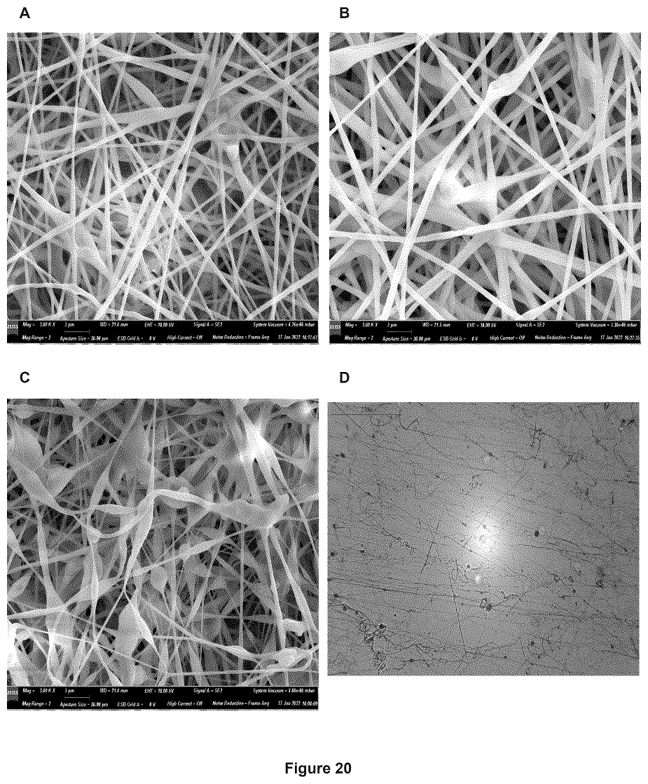

The PVA/PVP-based dry cream was examined by scanning electron microscopy. Image shown in FIG. 20C.

### Example 11-2: Gelatin

Conditions: Nozzle—22 G; Flow rate—0.6 mL/h, Voltage—27 kV; Distance—20 cm; plate collector

### Composition of Oil-In-Water Emulsion:

TABLE 7
Composition of gelatin-based dry cream

| Ingredients | Amount | |
|---|---|---|
| Castor oil | 0.1 | g |
| Gelatin (Sigma Aldrich, Ph Eur grade) | 2.5 | g |
| Glacial acetic acid | 4.0 | mL |
| MQ water | 6.0 | mL |

### Example 11-3: PVA/HPMC

Conditions: Nozzle—22 G; Flow rate—1 mL/h, Voltage—24 kV; Distance—20 cm; plate collector

### Composition of Oil-In-Water Emulsion:

TABLE 8
Composition of PVA/HPMC-based dry cream

| Ingredients | Amount |
|---|---|
| Peppermint oil | 0.1 g |
| Tefose 63 (Gattefossé) | 0.6 g |
| Labrafil M 1944 CS (Gattefossé) | 0.3 g |
| PVA (MW 85000-120000, 88% hydrolyzed, ACROS) | 0.7 g |
| HPMC (grade 615, viscosity 15 mPas, Shin-Etsu Pharmacoat) | 0.3 g |
| MQ water | 10.0 g |

### Example 11-4: PVA/PEO

(a) Example with Tea Tree Oil:

Conditions: Nozzle—22 G; Flow rate—1.0 mL/h, Voltage—11-12 kV; Distance—15 cm; Plate collector

### Composition of Oil-in-Water Emulsion:

TABLE 9
Composition of PVA/PEO-based dry cream (tea tree oil)

| Ingredients | Amount |
|---|---|
| Tea Tree Oil | 0.1 g |
| Tefose 63 (Gattefossé) | 0.6 g |
| Labrafil M 1944 CS (Gattefossé) | 0.3 g |
| PVA (MW 85000-120000, 88% hydrolyzed, ACROS) | 0.7 g |
| PEO K30 (MW 300,000) | 0.3 g |
| MQ water | 10.0 g |

(b) Example with Turmeric Oil:

Conditions: Nozzle—22 G; Flow rate—1 mL/h, Voltage—12-13 kV; Distance—15 cm; plate collector

### Composition of Oil-in-Water Emulsion:

TABLE 10
Composition of PVA/PEO-based dry cream (turmeric oil)

| Ingredients | Amount |
|---|---|
| Turmeric oil | 0.1 g |
| Tefose 63 (Gattefossé) | 0.6 g |
| Labrafil M 1944 CS (Gattefossé) | 0.3 g |
| PVA (MW 85000-120000, 88% hydrolyzed, ACROS) | 0.7 g |
| PEO (MW 300,000) | 0.3 g |
| MQ water | 10.0 g |

### Measurement of Burst Strength Mechanical Strength:

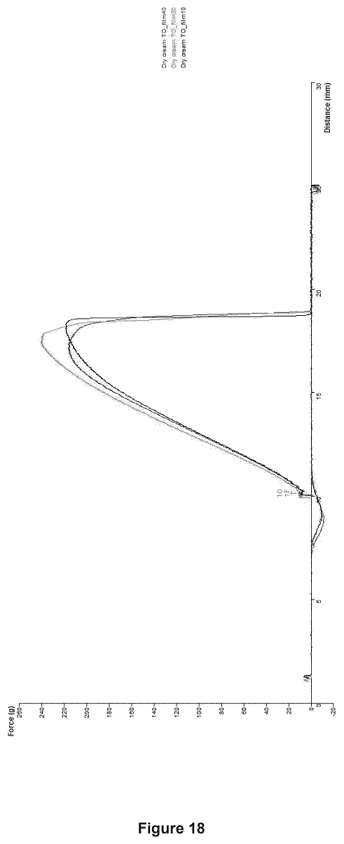

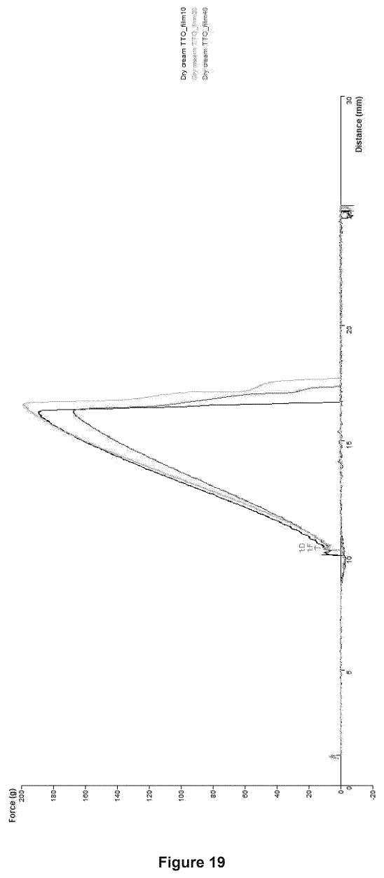

After a spinning time of 1 hour, the tea tree oil-based and the turmeric oil-based electrospun dry creams were subjected to burst strength testing. The results of these tests are shown below and in FIGS. 18 and 19 (with tissue as a comparative example).

TABLE 11
Comparison of thicknesses and burst strengths for tea tree
oil-based and the turmeric oil-based electrospun dry creams

| Film | Thickness (μm) | Burst strength (g) |
|---|---|---|
| Tissue | 142.1* ± 8.83 | 145.3** ± 34.5 |
| Tea tree oil dry cream | 57.1* ± 8.0 | 185.1 ± 16.1 |
| Turmeric oil dry cream | 97.0* ± 8.8 | 212.6** ± 11.8 |

*p < 0.05 for thickness;
**p < 0.05 for burst strength

Both the tea tree oil-based and the turmeric oil-based electrospun dry creams were found to have higher resistance to rupturing (burst strength) compared to the comparative tissue example. This is despite the tissue having higher thickness than either of the electrospun dry creams. This indicates that a relatively strong electrospun film could be produced from the oil-in-water emulsion.

### Fibre Morphology:

The tea tree oil-based and the turmeric oil-based electrospun dry creams were examined by scanning electron microscopy. Images are shown in FIGS. 20A and 20B.

The morphology of the fibres was substantially smooth with little beading. Without wishing to be bound by theory, it is postulated that the low amount of beading is due to the combination of PVA and PEO.

### Water Content:

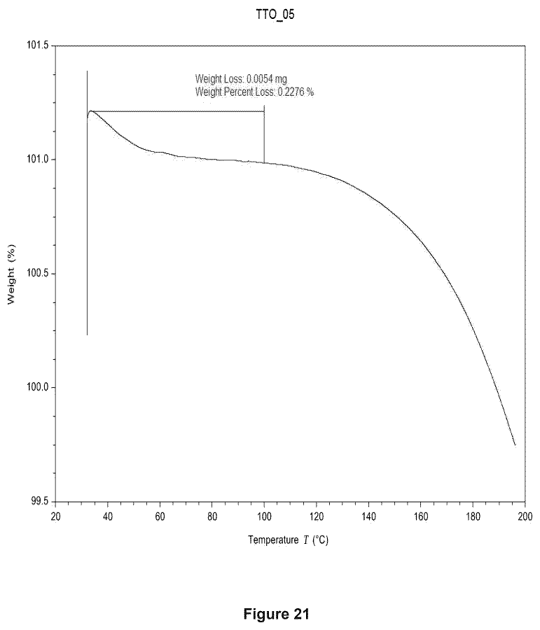

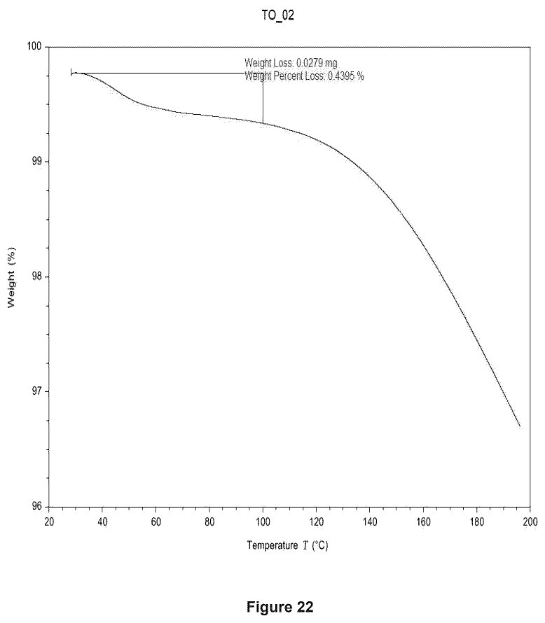

The tea tree oil-based and the turmeric oil-based electrospun dry creams were analysed for residual water content. Results of this testing is shown below and in FIGS. 21 and 22.

TABLE 12
Residual water contents of tea tree oil-based and
the turmeric oil-based electrospun dry creams

| Film | Water content (%) |
|---|---|
| Tea tree oil dry cream | 0.27% ± 0.06 |
| Turmeric oil dry cream | 0.41% ± 0.04 |

### Example 11-5: Pectin/PEO

Conditions: Nozzle—22 G; Flow rate—1 mL/h, Voltage—14-17 kV; Distance—10 cm; plate collector

### Composition of Oil-in-Water Emulsion:

TABLE 13
Composition of pectin/PEO-based dry cream

| Ingredients | Amount |
|---|---|
| Castor oil | 0.1 g |
| Tefose 63 (Gattefosse) | 0.6 g |
| Labrafil M 1944 CS (Gattefosse) | 0.3 g |
| Pectin (from apple) | 0.3 g |
| PEO (MW 300,000) | 0.7 g |
| MQ water | 10.0 g |

### Fibre Morphology:

The pectin/PEO-based dry cream was examined by optical microscopy. Image shown in FIG. 20D.

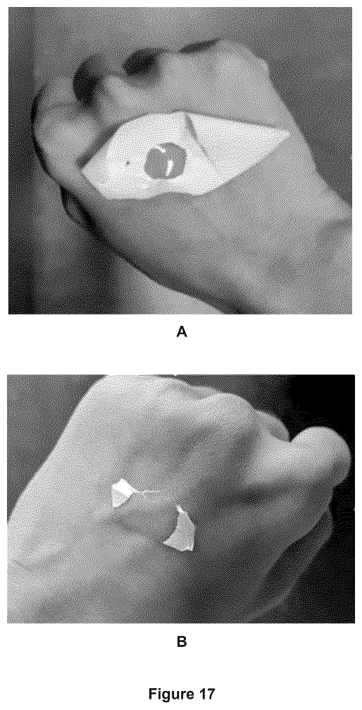

The dry creams were investigated for their response to wetting. In all cases, the dry cream formulations formed an emulsion when water was added (see e.g. PVA/PVP formulation—FIG. 17A; gelatin formulation—FIG. 17B). A milky liquid was formed and no visible large oil droplets were observed indicating no significant phase separation between the oil and water phases.

In conclusion, the present invention has identified that electrospun nanofiber/microfiber mats made according to the present invention have advantageous properties. They enable uptake of both hydrophilic and hydrophobic excipients and can carry many times their weight.

Products made according to the present invention can advantageously be stored in a dried state prior to application by the user. This can improve stability of bioactive agents during storage, leading to improved shelf life and/or higher bioactive activity. For example, mats made according to the present invention may have increased antioxidant stability. The dried mat may be re-hydrated in either commercial or premade solutions (aqueous, oil based or both).

Uptake and mechanical properties of mats according to the present invention could be varied by electrospinning parameters and polymer selection and molecular weight. It is therefore possible to modify properties depending on target application.

The mats of the present invention may advantageously be used in both a cosmetic or therapeutic application.

### REFERENCES

1. Baki, G. & Alexander, K. S. Introduction to Cosmetic Formulation and Technology. (John Wiley & Sons, 2015).
2. Goddard, E. D. & Gruber, J. V. Principles of Polymer Science and Technology in Cosmetics and Personal Care. (Taylor & Francis, 1999).
3. Panduranga Rao, K. Recent developments of collagen-based materials for medical applications and drug delivery systems. J. Biomater. Sci. Polym. Ed. 7, 623-645 (1996).
4. Haznedar, S. & Dortung, B. Preparation and in vitro evaluation of Eudragit microspheres containing acetazolamide. Int. J. Pharm. 269, 131-140 (2004).
5. Song, Y., Liu, L., Shen, H., You, J. & Luo, Y. Effect of sodium alginate-based edible coating containing different anti-oxidants on quality and shelf life of refrigerated bream (Megalobrama amblycephala). Food Control 22, 608-615 (2011).
6. Burnett, C. L. et al. Safety Assessment of PEGylated Oils as Used in Cosmetics. Int. J. Toxicol. 33, 13S-39S (2014).
7. Honigman, R. & Castle, D. J. Aging and cosmetic enhancement. Clin. Interv. Aging 1, 115-119 (2006).
8. Huang, Z.-M., Zhang, Y.-Z., Kotaki, M. & Ramakrishna, S. A review on polymer nanofibers by electrospinning and their applications in nanocomposites. Compos. Sci. Technol. 63, 2223-2253 (2003).
9. Balasundram, N., Sundram, K. & Samman, S. Phenolic compounds in plants and agriindustrial by-products: Antioxidant activity, occurrence, and potential uses. Food Chem. 99, 191-203 (2006).
10. Millqvist, E., Bengtsson, U. & Löwhagen, O. Combining a β2-agonist with a face mask to prevent exercise-induced bronchoconstriction. Allergy 55, 672-675 (2000).
11. Reneker, D. H. & Chun, I. Nanometre diameter fibers of polymer, produced by electrospinning. Nanotechnology 7, 216-223 (1996).
12. Hu, C., Gong, R. H. & Zhou, F. L. Electrospun Sodium Alginate/Polyethylene Oxide Fibers and Nanocoated Yarns. Int. J. Polym. Sci. 2015, 1-12 (2015).
13. Yuan, X., Zhang, Y., Dong, C. & Sheng, J. Morphology of ultrafine polysulfone fibers prepared by electrospinning. Polym. Int. 53, 1704-1710 (2004).
14. Beachley, V. & Wen, X. Effect of electrospinning parameters on the nanofiber diameter and length.
   Mater. Sci. Eng. C 29, 663-668 (2009).
15. Ying, Q., Lili, Z., Haixiang, S. & Peng, L. PAN/PS electrospun fibers for oil spill cleanup. IOP Conf.
   Ser. Mater. Sci. Eng. 62, 12032 (2014).
16. Qiao, Y., Zhao, L., Li, P., Sun, H. & Li, S. Electrospun polystyrene/polyacrylonitrile fiber with high oil sorption capacity. J. Reinf. Plast. Compos. 33, 1849-1858 (2014).
17. SUNI, S., KOSUNEN, A. L. & ROMANTSCHUK, M. Microbially Treated Peat-Cellulose Fabric as a Biodegradable Oil-Collection Cloth. J. Environ. Sci. Heal. Part A 41, 999-1007 (2006).
18. Lin, J. et al. Co-axial electrospun polystyrene/polyurethane fibers for oil collection from water surface. Nanoscale 5, 2745-55 (2013).
19. Lin, J. et al. Nanoporous polystyrene fibers for oil spill cleanup. Mar. Pollut. Bull. 64, 347-352 (2012).
20. Pillay, V., Dangor, C. M., Govender, T., Moopanar, K. R. & Hurbans, N. Ionotropic gelation: Encapsulation of indomethacin in calcium alginate gel discs. J. Microencapsul. 15, 215-226 (1998).
21. Shenoy, S. L., Bates, W. D., Frisch, H. L. & Wnek, G. E. Role of chain entanglements on fiber formation during electrospinning of polymer solutions: good solvent, non-specific polymer-polymer interaction limit. Polymer (Guildf). 46, 3372-3384 (2005).
22. Kakade, M. V et al. Electric Field Induced Orientation of Polymer Chains in Macroscopically Aligned Electrospun Polymer Nanofibers. J. Am. Chem. Soc. 129, 2777-2782 (2007).
23. Lin, J. et al. Facile control of intra-fiber porosity and inter-fiber voids in electrospun fibers for selective adsorption. Nanoscale 4, 5316 (2012).
24. Watanabe, K., Nakamura, T., Kim, B.-S. & Kim, I.-S. Effect of organic solvent on morphology and mechanical properties of electrospun syndiotactic polypropylene nanofibers. Polym. Bull. 67, 2025-2033 (2011).
25. Kim, J. I., Hwang, T. I., Aguilar, L. E., Park, C. H. & Kim, C. S. A Controlled Design of Aligned and Random Nanofibers for 3D Bi-functionalized Nerve Conduits Fabricated via a Novel Electrospinning Set-up. Sci. Rep. 6, 23761 (2016).
26. Carlisle, C. R. et al. The mechanical properties of individual, electrospun fibrinogen fibers. Biomaterials 30, 1205-1213 (2009).
27. Baker, S. et al. The mechanical properties of dry, electrospun fibrinogen fibers. Mater. Sci. Eng. C 32, 215-221 (2012).
28. Sheth, N. S. & Mistry, R. B. Formulation and evaluation of transdermal patches and to study permeation enhancement effect of eugenol. J. Appl. Pharm. Sci. 1, 96-101 (2011).
29. Pegoraro, C., MacNeil, S. & Battaglia, G. Transdermal drug delivery: from micro to nano. Nanoscale 4, 1881 (2012).
30. Marques, M. R. C., Loebenberg, R. & Almukainzi, M. Simulated Biological Fluids with Possible Application in Dissolution Testing. Dissolution Technol. 18, 15-28 (2011).
31. PermeGear, I. Parts of a Franz Cell. (2017).
32. TOPICAL DELIVERY—The Importance of the Right Formulation in Topical Drug Development; Drug Development & Delivery January/February 2015.
33. Benson, H. A. E, Topical and Transdermal Drug Delivery: From Simple Potions to Smart Technologies, Current Drug Delivery, 2019, 16, 444-460.
34. KR20110077792
35. US20110229551
36. WO2014098764
37. WO2009031620
38. Loftsson T, Brewster M E. Pharmaceutical applications of cyclodextrins: basic science and product development. Journal of Pharmacy and Pharmacology. 2010; 62(11):1607-21.
39. Kumar P, Singh C. A Study on Solubility Enhancement Methods for Poorly Water Soluble Drugs. American Journal of Pharmacological Sciences. 2013; 1(4):67-73.
40. Pouton C W. Formulation of poorly water-soluble drugs for oral administration: Physicochemical and physiological issues and the lipid formulation classification system. European Journal of Pharmaceutical Sciences. 2006; 29(3-4):278-87.
41. Leuner C, Dressman J. Improving drug solubility for oral delivery using solid dispersions. European Journal of Pharmaceutics and Biopharmaceutics. 2000; 50(1):47-60.
42. Gullapalli R P. Soft gelatin capsules (softgels). Journal of Pharmaceutical Sciences. 2010; 99(10):4107-48.
43. Masmoudi H, Dr\tilde{e}au Y L, Piccerelle P, Kister J. The evaluation of cosmetic and pharmaceutical emulsions aging process using classical techniques and a new method: FTIR. International Journal of Pharmaceutics. 2005; 289(1-2):117-31.
44. Patel A R, Rajarethinem P S, Cludts N, Lewille B, De Vos W H, Lesaffer A, et al. Biopolymer-Based Structuring of Liquid Oil into Soft Solids and Oleogels Using Water-Continuous Emulsions as Templates. Langmuir. 2015; 31(7):2065-73.
45. Amar-Yuli I, Adamcik J, Lara C, Bolisetty S, Vallooran J J, Mezzenga R. Templating effects of lyotropic liquid crystals in the encapsulation of amyloid fibrils and their stimuli-responsive magnetic behavior. Soft Matter. 2011; 7(7):3348-57.
46. Wilde P, Mackie A, Husband F, Gunning P, Morris V. Proteins and emulsifiers at liquid interfaces. Advances in Colloid and Interface Science. 2004; 108-109(0):63-71.
47. Osborn H T, Akoh C C. Structured Lipids-Novel Fats with Medical, Nutraceutical, and Food Applications. Comprehensive Reviews in Food Science and Food Safety. 2002; 1(3):110-20.
48. Rogers M A. Novel structuring strategies for unsaturated fats—Meeting the zero-trans, zero-saturated fat challenge: A review. Food Research International. 2009; 42(7):747-53.
49. Turnbull D, Fisher J C. Rate of Nucleation in Condensed Systems. The Journal of Chemical Physics. 1949; 17(1):71-3.
50. Marangoni A. Organogels: An Alternative Edible Oil-Structuring Method. J Am Oil Chem Soc. 2012; 89(5):749-80.
51. Romoscanu A I, Mezzenga R. Emulsion-Templated Fully Reversible Protein-in-Oil Gels. Langmuir. 2006; 22(18):7812-8.
52. Mezzenga R, Ulrich S. Spray-Dried Oil Powder with Ultrahigh Oil Content. Langmuir. 2010; 26(22):16658-61.
53. Reneker D H, Yarin A L. Electrospinning jets and polymer nanofibers. Polymer. 2008; 49(10):2387-425.
54. Bhardwaj N, Kundu S C. Electrospinning: A fascinating fiber fabrication technique. Biotechnology Advances. 2010; 28(3):325-47.
55. Huang Z-M, Zhang Y Z, Kotaki M, Ramakrishna S. A review on polymer nanofibers by electrospinning and their applications in nanocomposites. Composites Science and Technology. 2003; 63(15):2223-53.
56. Lu P, Ding B. Applications of Electrospun Fibers. Recent Patents on Nanotechnology. 2008; 2(3):169-82.
57. Doshi J, Reneker D H. Electrospinning process and applications of electrospun fibers. Journal of Electrostatics. 1995; 35(2-3):151-60.
58. Lin J, Tian F, Shang Y, Wang F, Ding B, Yu J, et al. Co-axial electrospun polystyrene/polyurethane fibers for oil collection from water surface. Nanoscale. 2013; 5(7):2745-55.
59. Zhu H, Qiu S, Jiang W, Wu D, Zhang C. Evaluation of Electrospun Polyvinyl Chloride/Polystyrene Fibers As Sorbent Materials for Oil Spill Cleanup. Environmental Science & Technology. 2011; 45(10):4527-31.
60. Yang Z, Nollenberger K, Albers J, Craig D, Qi S. Microstructure of an Immiscible Polymer Blend and Its Stabilization Effect on Amorphous Solid Dispersions. Molecular Pharmaceutics. 2013; 10(7):2767-80.
61. Tipduangta P, Belton P, Fábián L, Wang L Y, Tang H, Eddleston M, et al. Electrospun Polymer Blend Nanofibers for Tunable Drug Delivery: The Role of Transformative Phase Separation on Controlling the Release Rate. Molecular Pharmaceutics. 2015.
62. Barthel M J, Schacher F H, Schubert U S. Poly(ethylene oxide) (PEO)-based ABC triblock terpolymers—synthetic complexity vs. application benefits. Polymer Chemistry. 2014; 5(8):2647-62.
63. Karavas E, Georgarakis E, Bikiaris D. Application of PVP/HPMC miscible blends with enhanced mucoadhesive properties for adjusting drug release in predictable pulsatile chronotherapeutics. European Journal of Pharmaceutics and Biopharmaceutics. 2006; 64(1):115-26.
64. Oliveira J E, Mattoso L H C, Orts W J, Medeiros E S. Structural and Morphological Characterization of Micro and Nanofibers Produced by Electrospinning and Solution Blow Spinning: A Comparative Study. Advances in Materials Science and Engineering. 2013; 2013:14.
65. Ashara K C, Paun J S, Soniwala M M, Chavada J R, Mori N M. Micro-emulsion based emulgel: a novel topical drug delivery system. Asian Pacific Journal of Tropical Disease. 2014; 4, Supplement 1:S27-S32.
66. Khullar R, Kumar D, Seth N, Saini S. Formulation and evaluation of mefenamic acid emulgel for topical delivery. Saudi Pharmaceutical Journal. 2012; 20(1):63-7.
67. Stanos S P. Topical Agents for the Management of Musculoskeletal Pain. Journal of Pain and Symptom Management. 33(3):342-55.
68. Abdelwahab O. Assessment of raw luffa as a natural hollow oleophilic fibrous sorbent for oil spill cleanup. Alexandria Engineering Journal. 2014; 53(1):213-8.

1. A dry cream for delivery of oil(s) to a user, said dry cream comprising a mat of electrospun hydrophilic polymeric fibers containing said oil(s).

2. The dry cream according to claim 1, wherein the dry cream comprises less than 2.0 wt % of water, less than 1.9 wt % of water, less than 1.8 wt % of water, less than 1.7 wt % of water, less than 1.6 wt % of water, less than 1.5 wt % of water, less than 1.4 wt % of water, less than 1.3 wt % of water, less than 1.2 wt % of water, less than 1.1 wt % of water, less than 1.0 wt % of water, less than 0.9 wt % of water, less than 0.8 wt % of water, less than 0.7 wt % of water, less than 0.6 wt % of water, less than 0.5 wt % of water, less than 0.4 wt % of water, less than 0.3 wt % of water, less than 0.2 wt % of water, less than 0.1 wt % of water, less than 0.09 wt % of water, less than 0.08 wt % of water, less than 0.07 wt % of water, less than 0.06 wt % of water, less than 0.05 wt % of water, less than 0.04 wt % of water, less than 0.03 wt % of water, less than 0.02 wt % of water, or less than 0.01 wt % of water, based on a total weight of the dry cream.

3. The dry cream according to claim 1 or claim 2, wherein the total polymeric fiber content is about 40 wt % to about 99 wt %, about 42 wt % to about 98 wt %, about 44 wt % to about 97 wt %, about 46 wt % to about 96.5 wt %, about 48 wt % to about 96.5 wt %, or about 50 wt % to about 96.5 wt %, based on a total weight of the dry cream.

4. The dry cream according to any one of claims 1 to 3, wherein the total oil content of the dry cream is about 1 wt % to about 35 wt %, about 1 wt % to about 30 wt %, about 1 wt % to about 25 wt %, about 1 wt % to about 20 wt %, about 1 wt % to about 15 wt %, about 1 wt % to about 10 wt %, about 2 wt % to about 8 wt %, about 3 wt % to about 6 wt %, or about 3.5 wt % to about 5 wt %, based on a total weight of the dry cream.

5. The dry cream according to any one of claims 1 to 4, wherein a w/w ratio of the total oil content to the total polymeric fiber content is from about 1:5 to about 1:50, about 1:6 to about 1:45, about 1:7 to about 1:40, about 1:8 to about 1:35, about 1:9 to about 1:30, or about 1:10 to about 1:25.

6. The dry cream according to any one of claims 1 to 5, wherein the dry cream comprises one or more surfactant(s).

7. The dry cream according to claim 6, wherein a w/w ratio of the total polymeric fiber content to the total surfactant content may be from about 1:5 to about 1:0.5, about 1:4 to about 1:0.6, about 1:3 to about 1:0.7, about 1:2 to about 1:0.8, or about 1:1 to about 1:0.9.

8. The dry cream according to claim 6 or claim 7, wherein a w/w ratio of the total oil content to the total surfactant content may be from about 1:2 to about 1:20, about 1:3 to about 1:18, about 1:4 to about 1:16, about 1:5 to about 1:14, about 1:6 to about 1:12, about 1:7 to about 1:10, or about 1:8 to about 1:9.

9. A topical delivery system for delivery of bioactive agent(s) to a user, said topical delivery system comprising a mat of electrospun hydrophilic polymeric fibers containing said bioactive agent(s), wherein said mat is configured to substantially retain its shape on a mounting surface when exposed to a wetting agent but disintegrate under mechanical agitation to form a cream or gel.

10. The dry cream or topical delivery system according to any one of claims 1 to 9, wherein the polymer comprises a synthetic polymer and/or a natural polymer.

11. The dry cream or topical delivery system according to claim 10, wherein the polymer is a synthetic polymer, optionally selected from poly(N-isopropylacrylamide) (PNIPAM), polyacrylamide (PAM), poly(2-oxazoline), polyethylenimine (PEI), poly(acrylic acid), polymethacrylate (PMA) and other acrylic polymers, poly(ethylene glycol) (PEG), poly(ethylene oxide) (PEO), poly(vinyl alcohol) (PVA) and copolymers, poly(vinylpyrrolidone) (PVP) and copolymers, polyelectrolytes, cucurbit[n]uril hydrate, HPMC, HPMCAS, and combinations thereof.

12. The dry cream or topical delivery system according to claim 10, wherein the polymer is a natural polymer, optionally selected from proteins and carbohydrates; said proteins optionally selected from collagen, gelatin, elastin, silk fibroin, zein, Aloe vera, soy proteins, sodium alginate (SA); said carbohydrates optionally selected from collagen, hyaluronic acid, chitosan, alginates, heparin, pectin, cellulose-based derivatives, starch and modified starch, xylan, xanthan gum and other gums.

13. The dry cream or topical delivery system of any one of claims 1 to 12, wherein said fibers have an average thickness of around 20 nm to 100 μm, around 20 nm to 50 μm, around 20 nm to 20 μm, around 20 nm to 15 μm, around 20 nm to 10 μm, around 50 nm to 10 μm, around 100 nm to 10 μm, around 100 nm to 5 μm, around 150 nm to 5 μm, around 150 nm to 3 μm, around 150 nm to 2 μm, around 150 nm to 1 μm, around 150 nm to 900 nm, around 150 nm to 800 nm, around 150 nm to 700 nm, around 100 nm to 600 nm, around 150 nm to 400 nm, around 200 nm to 400 nm, around 250 nm to around 350 nm; around 50 nm, around 100 nm, around 150 nm, around 200 nm, around 250 nm, around 300 nm or around 350 nm.

14. The dry cream or topical delivery system of any one of claims 1 to 13, wherein said mat has a mean pore size of about 0.1 μm to about 2 μm, about 0.2 μm to about 1.5 μm, about 0.3 μm to about 1.2 μm, about 0.4 μm to about 1 μm, about 0.5 μm to about 0.9 μm, about 0.1 μm to about 1 μm, about 0.2 μm to about 1 μm, about 0.3 μm to about 1 μm, about 0.4 μm to about 1 μm, or about 0.5 μm to about 1 μm.

15. The dry cream or topical delivery system of any one of claims 1 to 14, wherein said mat has a total pore volume (TPV) of about 0.1 cc/g or above, about 0.2 cc/g or above, about 0.3 cc/g or above, about 0.4 cc/g or above, about 0.5 cc/g or above, or about 0.6 cc/g or above.

16. The dry cream or topical delivery system of any one of claims 1 to 15, wherein said topical delivery system is for delivery to the skin or to the mucous membranes.

17. The topical delivery system of any one of claims 9 to 16, wherein said topical delivery system is a mask, optionally a facemask.

18. The topical delivery system according to any one of claims 9 to 17, wherein the polymer is a high molecular weight (HMW) polymer.

19. The topical delivery system according to claim 18, wherein the polymer has a weight average molecular weight of 300 KDa or above, 320 KDa or above, 340 KDa or above, 360 KDa or above, 380 KDa or above, 400 KDa or above, 420 KDa or above, 440 KDa or above, 460 KDa or above, 480 KDa or above, 500 KDa or above, 520 KDa or above, 540 KDa or above, 560 KDa or above, 580 KDa or above, 600 KDa or above, 620 KDa or above, 640 KDa or above, 660 KDa or above, 680 KDa or above, or 700 KDa or above.

20. The topical delivery system according to any one of claims 9 to 19, wherein said topical delivery system comprises a backing layer disposed on said mat.

21. A method of topical delivery comprising applying a dry cream or topical delivery system according to any one of claims 1 to 20 to the user.

22. The method according to claim 21, wherein said mat is applied to the user after wetting, or wherein said mat is applied to the user after wetting.

23. The method according to claim 21 or claim 22, wherein said mat is wetted with a non-polar medium, for example oil; a polar medium, for example water; or an emulsion medium.

24. A dry cream or topical delivery system as claimed in any one of claims 1 to 20, for use as a medicament.

25. A cosmetic method comprising applying a dry cream or topical delivery system as claimed in any one of claims 1 to 20 to a user.
